SEQUENCE LISTING (12 sequences)

SEQ ID NO: 1 - protein, 218 aa, Mus musculus
DIVLTQSPASLAVSLGQRATISCKASESVDNYGKSLMHWYQQKPGQSPKLLIYRASNLESGIPARFSGSGSRTDFTLTINPVEADDVATYYCQQSNEDPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 215 aa, Mus musculus
EVKLVESGGGLVKPGGSLKLSCAASGFTFISYAMSWVRQTPEKRLEWVASISSGGNTYYPDSVKGRFTISRDNARNILYLQMSSLRSEDTAMYYCARLDGYYFGFAYWGQGTLVAVSAAKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKI

SEQ ID NO: 3 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 4 - protein, 113 aa, Homo sapiens
PGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 5 - DNA, 3560 bp, Artificial Sequence
  Synthetically generated oligonucleotide
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggcagcattcaaagcagaaggctttggggtgtgtgatacgaaacgaagcattggccgtaagtgcgattccggattagctgccaatgtgccaatcgcggggggttttcgttcaggactacaactgccacacaccaccaaagctaactgacaggagaatccagatggatgcacaaacacgccgccgcgaacgtcgcgcagagaaacaggctcaatggaaagcagcaaatcccctgttggttggggtaagcgcaaaaccagttccgaaagatttttttaactataaacgctgatggaagcgtttatgcggaagaggtaaagcccttcccgagtaacaaaaaaacaacagcataaataaccccgctcttacacattccagccctgaaaaagggcatcaaattaaaccacacctatggtgtatgcatttatttgcatacattcaatcaattgttatccaagaaggagatatacatatgggtccagttccaccatctactgctctgcgtgaactgattgaagaactggttaacatcacccagaaccagaaagctccgctgtgtaacggttccatggtttggtccatcaacctgaccgctggtatgtactgtgcagctctggaatccctgatcaacgtttctggttgctctgctatcgaaaaaacccagcgtatgctgtctggtttctgcccgcacaaagtttccgctggtcagttctcctctctgcacgttcgtgacaccaaaatcgaagttgctcagttcgtaaaagacctgctgctgcacctgaaaaaactgttccgtgaaggtcgtttcaactaataatctagagtcgacctgcagtaatcgtacagggtagtacaaataaaaaaggcacgtcagatgacgtgccttttttcttgtgagcagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 6 - DNA, 118 bp, Artificial Sequence
  Synthetically generated oligonucleotide
tatgggtccagttccaccatctactgctctgcgtgaactgattgaagaactggttaacatcacccagaaccagaaagctccgctgtgtaacggttccatggtttggtccatcaacctg

SEQ ID NO: 7 - DNA, 123 bp, Artificial Sequence
  Synthetically generated oligonucleotide
cagcggtcaggttgatggaccaaaccatggaaccgttacacagcggagctttctggttctgggtgatgttaaccagttcttcaatcagttcacgcagagcagtagatggtggaactggaccca

SEQ ID NO: 8 - DNA, 120 bp, Artificial Sequence
  Synthetically generated oligonucleotide
accgctggtatgtactgtgcagctctggaatccctgatcaacgtttctggttgctctgctatcgaaaaaacccagcgtatgctgtctggtttctgcccgcacaaagtttccgctggtcag

SEQ ID NO: 9 - DNA, 121 bp, Artificial Sequence
  Synthetically generated oligonucleotide
gaggagaactgaccagcggaaactttgtgcgggcagaaaccagacagcatacgctgggttttttcgatagcagagcaaccagaaacgttgatcagggattccagagctgcacagtacatac

SEQ ID NO: 10 - DNA, 109 bp, Artificial Sequence
  Synthetically generated oligonucleotide
ttctcctctctgcacgttcgtgacaccaaaatcgaagttgctcagttcgtaaaagacctgctgctgcacctgaaaaaactgttccgtgaaggtcgtttcaactaataat

SEQ ID NO: 11 - DNA, 105 bp, Artificial Sequence
  Synthetically generated oligonucleotide
ctagattattagttgaaacgaccttcacggaacagttttttcaggtgcagcagcaggtcttttacgaactgagcaacttcgattttggtgtcacgaacgtgcaga

SEQ ID NO: 12 - protein, 427 aa, Homo sapiens
MEWPARLCGLWALLLCAGGGGGGGGAAPTETQPPVTNLSVSVENLCTVIWTWNPPEGASSNCSLWYFSHFGDKQDKKIAPETRRSIEVPLNERICLQVGSQCSTNESEKPSILVEKCISPPEGDPESAVTELQCIWHNLSYMKCSWLPGRNTSPDTNYTLYYWHRSLEKIHQCENIFREGQYFGCSFDLTKVKDSSFEQHSVQIMVKDNAGKIKPSFNIVPLTSRVKPDPPHIKNLSFHNDDLYVQWENPQNFISRCLFYEVEVNNSQTETHNVFYVQEAKCENPEFERNVENTSCFMVPGVLPDTLNTVRIRVKTNKLCYEDDKLWSNWSQEMSIGKKRNSTLYITMLLIVPVIVAGAIIVLLLYLKRLKIIIFPPIPDPGKIFKEMFGDQNDDTLHWKKYDIYEKQTKEETDSVVLIENLKKASQ

(19) United States
(12) Patent Application Publication
(10) Pub. No.: US 2007/0048785 A1
(43) Pub. Date: Mar. 1, 2007

(54) Anti-IL-13 antibodies and complexes

(76) Inventors: Laura Long Lin, Weston, MA (US); Kevin D. Parris, Auburndale, MA (US); Amy Sze Pui Tam, Medford, MA (US); Xiang-Yang Tan, Reading, MA (US); Tania Shane, Newton, MA (US); John Dumas, Arlington, MA (US); James M. Wilhelm, Boston, MA (US); Mark Stahl, Lexington, MA (US); Lidia Mosyak, Newton, MA (US); Zhixiang Hu, Spring, TX (US)

Correspondence Address: FISH & RICHARDSON P.C., P.O BOX 1022, MINNEAPOLIS, MN, 55440-1022, US

(21) Appl. No.: 11/149,025

(22) Filed: Jun. 9, 2005

Related U.S. Application Data
(60) Provisional application No. 60/578,736, filed on Jun. 9, 2004.
(60) Provisional application No. 60/578,473, filed on Jun. 9, 2004.
(60) Provisional application No. 60/581,375, filed on Jun. 22, 2004.

(51) Int. Cl. C07K 16/28; G01N 33/53; G06F 19/00

(52) U.S. Cl. 435/7.1; 530/388.22; 702/19

(57) ABSTRACT

Anti-IL-13 antibodies, crystals of anti-IL-13 antibodies, IL-13 polypeptide/anti-IL-13 antibody complexes, crystals of IL-13 polypeptide/anti-IL-13 antibody complexes, IL-13Rα1 polypeptide/IL-13 polypeptide/anti-IL-13 antibody complexes, crystals of IL-13Rα1 polypeptide/IL-13 polypeptide/anti-IL-13 antibody complexes, and related methods and software systems are disclosed.

### RELATED APPLICATIONS

This application claims the benefit of U.S. Provisional Application No. 60/578,736, filed Jun. 9, 2004, U.S. Provisional Patent Application No. 60/578,473, filed Jun. 9, 2004, and U.S. Provisional Patent Application No. 60/581,375 filed Jun. 22, 2004. The contents of each of these applications are incorporated herein by reference in their entirety.

### TECHNICAL FIELD

The invention relates to anti-IL-13 antibodies, crystals of anti-IL-13 antibodies, IL-13 polypeptide/anti-IL-13 antibody complexes, crystals of IL-13 polypeptide/anti-IL-13 antibody complexes, IL-13Rα1 polypeptide/IL-13 polypeptide/anti-IL-13 antibody complexes, crystals of IL-13Rα 1 polypeptide/IL-13 polypeptide/anti-IL-13 antibody complexes, and related methods and software systems.

### BACKGROUND

Interleukin-13 (IL-13) is a pleiotropic cytokine involved in immune response conditions, such as atopy, asthma, allergy, and inflammatory response. The role of IL-13 in immune response is facilitated by its effect on cell-signaling pathways. For example, IL-13 can promote B cell proliferation, induce B cells to produce IgE, and down regulate the production of proinflammatory cytokines. IL-13 can also increase expression of VCAM-1 on endothelial cells, and enhance expression of class II MHC antigens and various adhesion molecules on monocytes.

IL-13 function is mediated through an interaction with its receptor on hematopoietic and other cell types. The human IL-13 receptor (IL-13R) is a heterodimer that includes the interleukin-4 receptor α chain, IL-4Rα, and the IL-13 binding chain, IL-13Rα1. The association of IL-13 with its receptor induces the activation of STAT6 (signal transducer and activation of transcription 6) and JAK1 (Janus-family kinase) through a binding interaction with the IL-4Rα chain. IL-13Rα2, which may be found on the cell surface or in soluble form in the circulation, binds to IL-13 with high affinity but does not mediate cellular responses to IL-13. It is thought to function as a decoy receptor.

### SUMMARY

In one aspect, the invention features a crystalline antibody. The crystalline antibody is an anti-IL-13 antibody or a Fab fragment of an anti-IL-13 antibody.

In another aspect, the invention features a crystalline composition that includes an antibody. The antibody is an anti-IL-13 antibody or a Fab fragment of an anti-IL-13 antibody.

In a further aspect, the invention features a crystalline complex that includes an IL-13 polypeptide and an antibody. The antibody is an anti-IL-13 antibody or a Fab fragment of an anti-IL-13 antibody.

In another aspect, the invention features a crystalline complex that includes an IL-13Rα1 polypeptide and an IL-13 polypeptide.

In yet another aspect, the invention features a method that includes using a three-dimensional model of an antibody to design an agent that interacts with an IL-13 polypeptide. The antibody is an anti-IL-13 antibody or a Fab fragment of an anti-IL-13 antibody.

In another aspect, the invention features a method that includes using a three-dimensional model of an IL-13 polypeptide to design an agent that interacts with the IL-13 polypeptide.

In another aspect, the invention features a method that includes using a three-dimensional model of an IL-13 polypeptide bound to an IL-13Rα1 polypeptide to design an agent that interacts with the IL-13 polypeptide.

In another aspect, the invention features a method that includes selecting an agent by performing rational drug design with a three-dimensional structure of a crystalline complex that includes an IL-13 polypeptide; contacting the agent with an IL-13 polypeptide; and detecting the ability of the agent to bind the IL-13 polypeptide.

In a further aspect, the invention features a method that includes contacting an IL-13 polypeptide with an antibody to form a composition; and crystallizing the composition to form a crystalline complex in which the antibody is bound to the IL-13 polypeptide. The antibody is an anti-IL-13 antibody or a Fab fragment of an anti-IL-13 antibody, and the crystalline complex can diffract X-rays to a resolution of at least about 3.5 Å.

In another aspect, the invention features a method that includes contacting an IL-13 polypeptide with an antibody and an IL-13Rα1 polypeptide to form a composition, and crystallizing the composition to form a crystalline complex in which the antibody and the IL-13Rα1 polypeptide are each bound to the IL-13 polypeptide. The antibody is an anti-IL-13 antibody or a Fab fragment of an anti-IL-13 antibody, and the crystalline complex can diffract X-rays to a resolution of at least about 3.5 Å.

In another aspect, the invention features a software system that includes instructions for causing a computer system to accept information relating to a structure of an IL-13 polypeptide bound to an antibody, the antibody including an anti-IL-13 antibody or a Fab fragment of an anti-IL-13 antibody. The instructions also cause the computer system to accept information relating to a candidate agent and to determine binding characteristics of the candidate agent to the IL-13 polypeptide. The determination of binding characteristics is based on the information relating to the structure of the IL-13 polypeptide and the information relating to the candidate agent.

In another aspect, the invention features a computer program residing on a computer readable medium. A plurality of instructions is stored on the computer readable medium. When the instructions are executed by one or more processors, the one or more processors will accept information relating to a structure of an IL-13 polypeptide bound to an antibody, the antibody being an anti-IL-13 polypeptide or a Fab fragment of an anti-IL-13 antibody; accept information relating to a candidate agent; and determine binding characteristics of the candidate agent to the IL-13 polypeptide. Determination of the binding characteristics is based on the information relating to the structure of the IL-13 polypeptide and the information relating to the candidate agent.

In another aspect, the invention features a method that includes accepting information relating to the structure of an IL-13 polypeptide bound to an antibody and modeling the binding characteristics of the IL-13 polypeptide with a candidate agent. The antibody is an anti-IL-13 antibody or a Fab fragment of an anti-IL-13 antibody. The method of accepting information and modeling the binding characteristics is implemented by a software system.

In another aspect, the invention features a computer program residing on a computer readable medium containing a plurality of instructions. When the instructions are executed by one or more processors, the one or more processors will accept information relating to the structure of an IL-13 polypeptide bound to an antibody, the antibody being an anti-IL-13 antibody or a Fab fragment of an anti-IL-13 antibody; and model the binding characteristics of the IL-13 polypeptide with a candidate agent.

In another aspect, the invention features a software system, that includes instructions for causing a computer system to accept information relating to the structure of an IL-13 polypeptide bound to an antibody, and model the binding characteristics of the IL-13 polypeptide with a candidate agent. The antibody is an anti-IL-13 antibody or a Fab fragment of an anti-IL-13 antibody.

In another aspect, the invention features a crystalline antibody. The antibody is capable of binding to a site of an IL-13 polypeptide to which an IL-4R polypeptide binds in vivo.

In a further aspect, the invention features a crystalline composition that includes an antibody capable of binding a site of an IL-13 polypeptide to which an IL-4R polypeptide binds in vivo.

In another aspect, the invention features a crystalline complex that includes an IL-13 polypeptide and an antibody. The antibody is capable of binding a site of an IL-13 polypeptide to which an IL-4R polypeptide binds in vivo.

In yet another aspect, the invention features a crystalline complex that includes an IL-13 polypeptide, an IL-13Ra1 polypeptide, and an antibody. The antibody is capable of binding a site of an IL-13 polypeptide to which an IL-4R polypeptide binds in vivo.

In another aspect, the invention features a method that includes using a three-dimensional model of an antibody to design an agent that interacts with an IL-13 polypeptide. The antibody is capable of binding a site of an IL-13 polypeptide to which an IL-4R polypeptide binds in vivo.

In another aspect, the invention features a method that includes contacting an IL-13 polypeptide with an antibody to form a composition; and crystallizing the composition to form a crystalline complex in which the antibody is bound to the IL-13 polypeptide. The antibody is capable of binding a site of an IL-13 polypeptide to which an IL-4R polypeptide binds in vivo, and the crystalline complex can diffract X-rays to a resolution of at least about 3.5 Å.

In yet another aspect, the invention features a method that includes contacting an IL-13 polypeptide with an antibody and an IL-13Rα1 polypeptide to form a composition, and crystallizing the composition to form a crystalline complex in which the antibody and the IL-13Rα1 polypeptide are each bound to the IL-13 polypeptide. The antibody is capable of binding a site of an IL-13 polypeptide to which an IL-4R polypeptide binds in vivo, and the crystalline complex can diffract X-rays to a resolution of at least about 3.5 Å.

In another aspect, the invention features a software system that includes instructions for causing a computer system to accept information relating to a structure of an IL-13 polypeptide bound to an antibody, accept information relating to a candidate agent, and determine binding characteristics of the candidate agent to the IL-13 polypeptide. The antibody is capable of binding a site of an IL-13 polypeptide to which an IL-4R polypeptide binds in vivo. The determination of binding characteristics of the candidate agent is based on the information relating to the structure of the IL-13 polypeptide and the information relating to the candidate agent.

In another aspect, the invention features a computer program residing on a computer readable medium containing a plurality of instructions. When the instructions are executed by one or more processors, the one or more processors will accept information relating to a structure of an IL-13 polypeptide bound to an antibody, accept information relating to a candidate agent; and determine the binding characteristics of the candidate agent to the IL-13 polypeptide. The antibody is capable of binding a site of an IL-13 polypeptide to which an IL-4R polypeptide binds in vivo. Determination of the binding characteristics of the candidate agent is based on the information relating to the structure of the IL-13 polypeptide and the information relating to the candidate agent

In another aspect, the invention features a method that includes accepting information relating to the structure of an IL-13 polypeptide bound to an antibody and modeling the binding characteristics of the IL-13 polypeptide with a candidate agent. The antibody is capable of binding a site of an IL-13 polypeptide to which an IL-4R polypeptide binds in vivo. The method of accepting information and modeling the binding characteristics is implemented by a software system.

In another aspect, the invention features a computer program residing on a computer readable medium containing a plurality of instructions. When the instructions are executed by one or more processors, the one or more processors will accept information relating to the structure of an IL-13 polypeptide bound to an antibody and model the binding characteristics of the IL-13 polypeptide with a candidate agent. The antibody is capable of binding a site of an IL-13 polypeptide to which an IL-4R polypeptide binds in vivo.

In another aspect, the invention features a software system that includes instructions for causing a computer system to accept information relating to the structure of an IL-13 polypeptide bound to an antibody and model the binding characteristics of the IL-13 polypeptide with a candidate agent. The antibody is capable of binding a site of an IL-13 polypeptide to which an IL-4R polypeptide binds in vivo.

In another aspect, the invention features a method of modulating IL-13 activity in a subject. The method includes using rational drug design to select an agent that is capable of modulating IL-13 activity, and administering a therapeutically effective amount of the agent to the subject.

In a further aspect, the invention features a method of treating a subject having a condition associated with IL-13 activity. The method includes using rational drug design to select an agent that is capable of effecting IL-13 activity, and administering a therapeutically effective amount of the agent to the subject.

In another aspect, the invention features a method of prophylactically treating a subject susceptible to a condition associated with IL-13 activity. The method includes determining that the subject is susceptible to the condition associated with IL-13 activity, using rational drug design to select an agent that is capable of effecting IL-13 activity, and administering a therapeutically effective amount of the agent to the subject.

Structural information of a polypeptide or a corresponding ligand can lead to a greater understanding of how the polypeptide functions in vivo. For example, knowledge of the structure of a protein or a corresponding ligand can reveal properties that facilitate the interaction of the protein with its ligands, including other proteins, antibodies, effector molecules (e.g., hormones), and nucleic acids. Structure based modeling can be used to identify ligands capable of interacting with an IL-13 polypeptide, thus eliminating the need for screening assays, which can be expensive and time-consuming. Structural information can also be used to direct the modification of a ligand known to interact with IL-13 to generate an alternative ligand with more desirable properties, such as tighter binding or greater specificity.

The study of the interaction between an anti-IL-13 antibody and an IL-13 polypeptide and between an IL-13 polypeptide and its receptor can facilitate the design or selection of ligands (e.g., drugs) for modulating the activity of IL-13 in vivo. Such studies can therefore be useful for designing therapeutic agents. Activity assays indicated that mAb13.2 blocked IL-13 function in vitro and in vivo (see Examples 1 and 2 below), including the use of an antibody to identify IL-13-binding agents capable of disturbing the normal function of the protein. Accordingly, it is believed that the crystal structures of the mAb13.2Fab fragment, the human IL-13/mAb13.2 Fab fragment complex, and the human IL-13Rα1 polypeptide/human IL-13/mAb13.2 Fab fragment complex (see Tables 10-12 below) can be useful for designing or identifying agents that can interact with IL-13 and the IL-13 receptor polypeptide, IL-13Rα1. Such agents may be useful in modulating the activity of IL-13 in immune response conditions, such as, for example, asthma (e.g., nonallergic asthma, or allergic asthma, which is sometimes referred to as chronic allergic airway disease), chronic obstructive pulmonary disorder (COPD), airway inflammation, eosinophilia, fibrosis and excess mucus production (e.g., cystic fibrosis, pulmonary fibrosis, and allergic rhinitis), inflammatory and/or autoimmune conditions of the skin (e.g., atopic dermatitis), inflammatory and/or autoimmune conditions of the gastrointestinal organs (e.g., inflammatory bowel disease (IBD) and/or Crohn's disease), liver (e.g., cirrhosis), inflammatory and/or autoimmune conditions of the blood vessels or connective tissue (e.g., scleroderma), and tumors or cancers (e.g., soft tissue or solid tumors), such as Hodgkin's lymphoma, glioblastoma, and lymphoma.

Other features and advantages of the invention will be apparent from the accompanying drawings and description, and from the claims. The contents of all references, pending patent applications and published patents, cited throughout this application are hereby expressly incorporated by reference. In case of conflict, the present application, including definitions, will control.

### DESCRIPTION OF DRAWINGS

FIG. 1A is the amino acid sequence of the light chain of the mAb13.2 Fab (fragment antigen binding) fragment (SEQ ID NO:1).

FIG. 1B is the amino acid sequence of the heavy chain of mAb13.2 Fab fragment (SEQ ID NO:2).

FIG. 2A is the amino acid sequence of full-length human IL-13 (Swiss-Prot Accession No. P35225) (SEQ ID NO:3). The signal peptide cleavage site is indicated by a slash. Alpha helices A, B, C, and D are underlined. Helix A is defined by amino acids 25-42; helix B is defined by amino acids 62-71; helix C is defined by amino acids 78-89; and helix D is defined by amino acids 112-127.

FIG. 2B is the amino acid sequence of human IL-13 (SEQ ID NO:4) following cleavage of the signal peptide. Alpha helices A, B, C, and D are underlined. Helix A is defined by amino acids 6-23; helix B is defined by amino acids 43-52; helix C is defined by amino acids 59-70; helix D is defined by amino acids 93-108.

FIG. 3 is a ribbon diagram illustrating the crystal structure of mAb13.2 Fab fragment (left) with the processed form of human IL-13 (right) (see FIG. 2B). The light chain of mAb13.2 Fab fragment is shown in dark shading, and the heavy chain in light shading. Helices A, B, C, and D of the IL-13 structure are indicated.

FIG. 4 is a graph illustrating the kinetic parameters of three different anti-IL-13 antibodies (mAb13.2, mAb13.4, and mAb13.9) binding to human IL-13 as determined by Biacore analyses. Kinetic constants for mAb13.2 are also shown.

FIG. 5 is a graph illustrating the binding of biotinylated mAb13.2 to recombinant and native human IL-13. ELISA plates were coated with anti-FLAG M2 antibody. The binding of FLAG-human IL-13 was detected with biotinylated mAb13.2 and streptavidin-peroxidase. This binding could be competed with native human IL-13 isolated from mitogen activated, Th2-skewed, cord blood mononuclear cells (triangles); and recombinant human IL-13 (diamonds). There was no detectable binding of recombinant murine IL-13 (circles) to mAb13.2.

FIG. 6 is a graph illustrating the effect of mAb13.2 and the known inhibitor rhuIL-13Rα2 on the bioactivity of human IL-13. “cpm” is the measure of ³H-thymidine taken up into TF1 cells grown in the presence of IL-13 and varying concentrations of mAb13.2 or rhuIL-13Rα2 (x-axis).

FIG. 7A is a graph illustrating the effect of recombinant human IL-13 and IL-4 on CD23 expression on CD11b+ monocytes. The monocytes were normal peripheral blood mononuclear cells (PBMCs) harvested from a healthy donor. The cells were treated overnight with 1 ng/mL recombinant human IL-13 or IL-4, then assayed for CD23 expression by flow cytometry.

FIG. 7B is a graph illustrating the effect of mAb13.2 on IL-13-induced CD23 expression on CD11b+ monocytes.

FIG. 7C is a graph illustrating the effect of mAb13.2 on IL-4-induced CD23 expression on CD11b+ monocytes.

FIG. 8 is a graph illustrating the effect of mAb13.2 on IL-13-dependent IgE production by human B cells. PBMC from a healthy donor were stimulated with PHA and IL-13. After 3 weeks, each well was assayed for IgE concentrations by ELISA. PHA+IL-13 increased the frequency of IgE-producing B cell clones. This effect was inhibited by mAb13.2, but not by an IL-13 specific nonneutralizing antibody (mAb13.8) or by control mouse IgG (msIgG).

FIG. 9A is a Western blot detecting phosphorylated STAT6 protein from HT-29 human epithelial cells treated with the indicated concentration of IL-13 for 30 min at 37° C.

FIG. 9B is a histogram from flow cytometry experiments that measured the level of cellular phosphorylated STAT6 protein following treatment with IL-13. The shift in phospho-STAT6 staining intensity upon treatment with IL-13 is indicated by the lightly shaded trace.

FIG. 9C is a panel of histograms from flow cytometry experiments that measured the level of cellular phosphorylated STAT6 protein following treatment with a sub-optimal concentration of human IL-13 and the indicated antibody. Cells treated with IL-13 and antibody are indicated by the bold trace. Shaded histograms indicate untreated cells. In addition to mAb13.2, an IL-13 specific nonneutralizing antibody (mAb13.8) and a control mouse IgG1 were also tested.

FIG. 10 is a graph demonstrating the percentage of eosinophils detected in BAL from Cynomolgus monkeys sensitized to Ascaris suum following lung segmental challenge with Ascaris antigen. Twenty-four hours before challenge, animals had been administered mAb13.2 i.v. (diamonds) or left untreated (circles). Triangles represent mAb13-2-treated and re-challenged with Ascaris at three months post-Ab administration. Eosinophils were detected by flow cytometry using depolarized side scatter analysis.

FIG. 11A is a graph showing that unlabeled mAb13.2 (diamonds) or mAb13.2 Fab fragments (circles) could compete for binding with biotinylated mAb13.2 in an ELISA assay. An “irrelevant antibody” (monoclonal antibody mAb13.8, which binds IL-13 but does not neutralize its activity) (asterisks) could not compete for binding. Competitor concentration is expressed as picomole (pM) antibody or Fab.

FIG. 11B is a graph showing that unlabeled mAb13.2 (diamonds) or mAb13.2 Fab fragment (circles) could compete for binding with biotinylated mAb13.2 in an ELISA assay. An “irrelevant antibody” (monoclonal antibody mAb13.8) (asterisks) could not compete for binding. Competitor concentration is expressed as picomole (PM) binding sites, assuming two binding sites per intact IgG and one binding site per Fab fragment.

FIG. 12A is a graph showing that mAb13.2 (diamonds) and mAb13.2 Fab fragment (circles) inhibited IL-13-dependent TF1 cell division. “Competitor concentration” is mAb13.2 and mAb13.2 Fab fragment concentration, and concentration is represented as pM competitor binding sites, assuming two binding sites per intact IgG and one binding site per Fab fragment.

FIG. 12B is a graph showing that mAb13.2 (diamonds) and mAb13.2 Fab fragment (circles) inhibited IL-13 CD23 expression on human PBMCs. Competitor concentration is mAb13.2 and mAb13.2 Fab fragment concentration, and the concentration is represented as pM competitor binding sites, assuming two binding sites per intact IgG and one binding site per Fab fragment.

FIG. 13 is the DNA sequence of the expression vector pAL-981 (SEQ ID NO:5), including a human IL-13 cDNA insert (hIL13coli). The cDNA sequence encoding IL-13 is underlined. Restriction sites Nde1 (nucleotide position 2722) and Xba1 (nucleotide position 3070) flank the cDNA sequence.

FIG. 14 is the amino acid sequence of human IL-13Rα1 (Swiss-Prot Accession No. P78552) (SEQ ID NO:12).

FIG. 15 is a ribbon diagram illustrating the structure of the mAb13.2 Fab/IL-13/IL-13Rα1 trimeric complex.

FIG. 16 is a ribbon diagram illustrating the interaction between IL-13 and Ig domain 1 of IL-13Rα1.

FIG. 17 is a ribbon diagram illustrating the interaction between IL-13 and Ig domain 3 of IL-13Rα1.

### DETAILED DESCRIPTION

The structure of the antigen binding fragment (Fab) of a murine monoclonal anti-IL-13 antibody, mAb13.2, was discovered by X-ray crystallography (see Table 10 below). The crystal structures of human IL-13 complexed with the mAb13.2 Fab fragment, and of human IL-13 complexed with both the mAb13.2 Fab fragment and an IL-13Rα1 polypeptide fragment were also discovered by X-ray crystallography (See Tables 11 and 12 below, respectively).

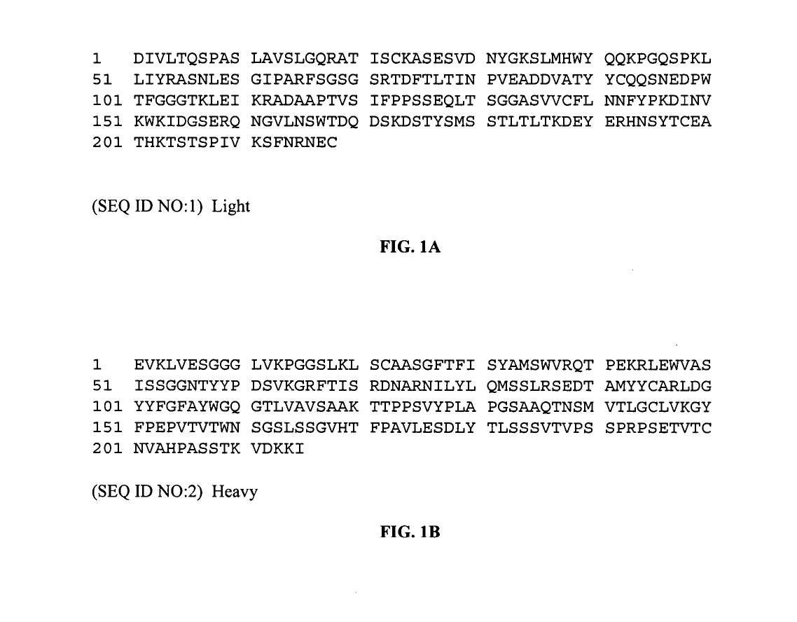

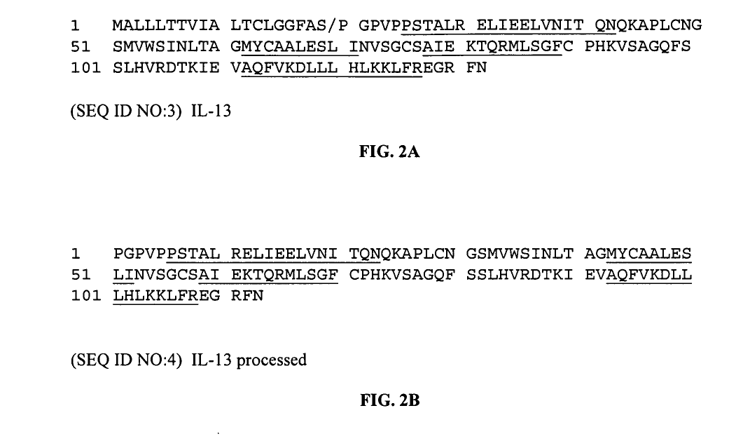

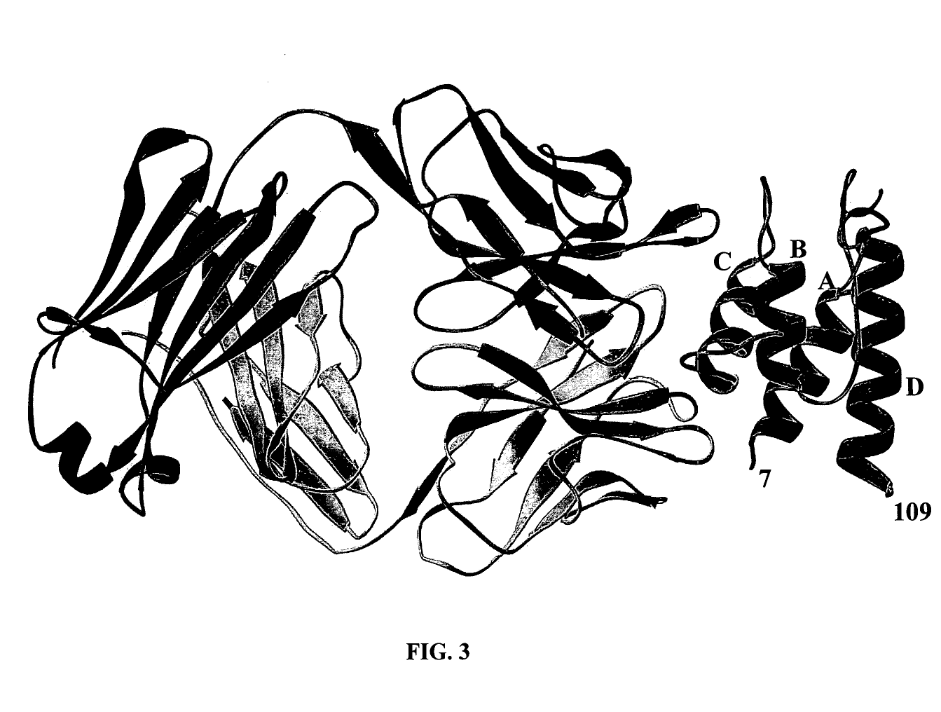

FIGS. 1A and 1B provide amino acid sequence information for the light and heavy chain polypeptides of the mAb13.2 Fab fragment. FIGS. 2A and 2B provide amino acid sequence information for human IL-13. FIG. 3 provides structural information for a crystal of a human IL-13/mAb13.2 Fab fragment complex. The mAb13.2 Fab fragment binds to the IL-4R (IL-4Rα) binding domain of human IL-13, which includes the amino acids Ser7, Thr8, Ala9, Glu12, Leu48, Glu49, Ile52, Asn53, Arg65, Met66, Ser68, Gly69, Phe70, Cys71, Pro72, His73, Lys74, and Arg86 as defined by SEQ ID NO:4.

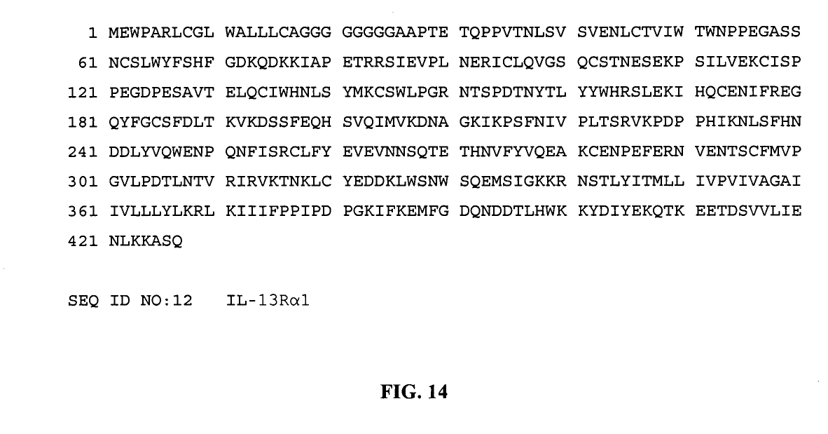

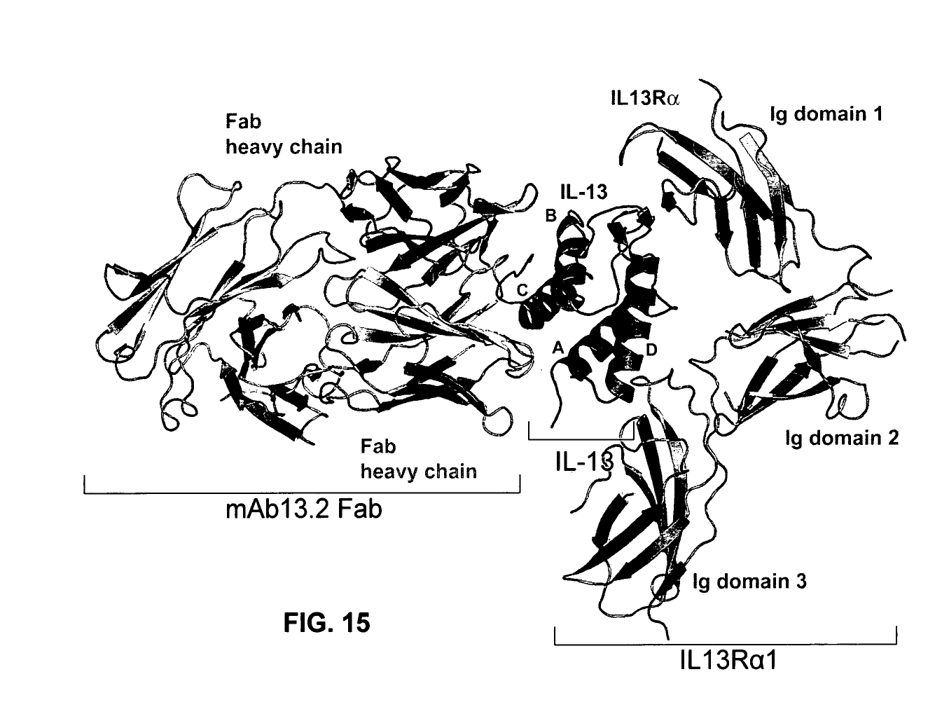

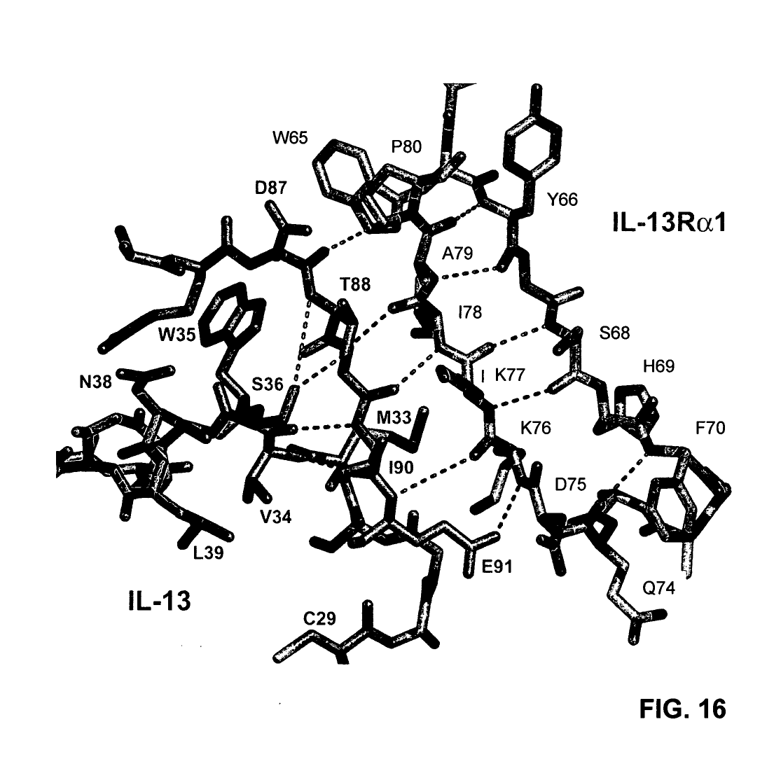

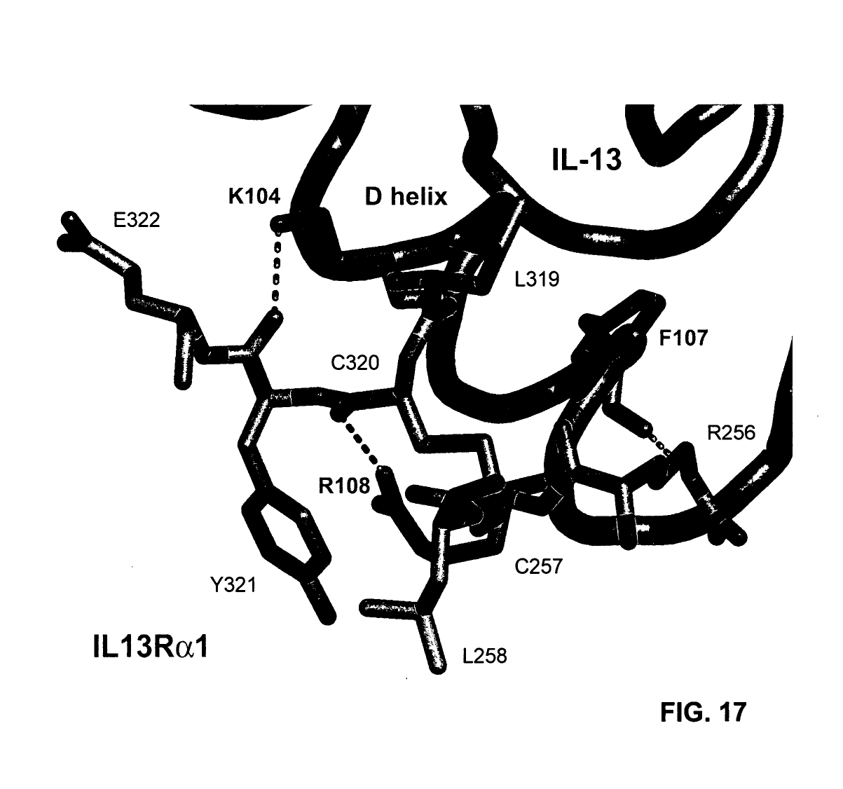

FIG. 14 provides amino acid sequence information for the human IL-13 receptor polypeptide, human IL-13Rα1. FIGS. 15, 16, and 17 provide structural information for a crystal of a human IL-13Rα1 polypeptide/human IL-13/mAb13.2 Fab fragment complex. In addition to the interaction described above between human IL-13 and the mAb13.2 Fab fragment, human IL-13 forms two contacts with the human IL-13Rα1 polypeptide, one with Ig domain 1 of the human IL-13Rα1 polypeptide, and a second with the Ig domain 3 of the human IL-13Rα1 polypeptide. The interaction with Ig domain 1 involves residues Thr88, Lys89, Ile90, and Glu91 of human IL-13 as defined by SEQ ID NO:4, and residues Lys76, Lys77, Ile78, and Ala79 of the human IL-13Rα1 polypeptide, as defined by SEQ ID NO:12 (see FIG. 16). The interaction with Ig domain 3 involves residues Arg11, Glu12, Leu13, Ile14, Glu15, Lys104, Lys105, Leu106, Phe107, and Arg108 of human IL-13 as defined by SEQ ID NO:4, and residues Ile254, Ser255, Arg256, Lys318, Cys320, and Tyr321 of the human IL-13Rα1 polypeptide as defined by SEQ ID NO:12 (see FIG. 17).

In general, a crystal of the mAb13.2 Fab fragment can be prepared as desired. Typically, the process includes first isolating the mAb13.2 Fab fragment, and then forming a crystal that contains that mAb13.2 Fab fragment. In some embodiments, a crystal containing the mAb13.2 Fab fragment can be prepared as follows. The intact antibody is cleaved with an appropriate proteolytic enzyme (e.g., papain), and the mAb13.2 Fab fragment is isolated from the Fc (Fragment crystallizable) fragment. The isolated mAb13.2 Fab fragment is disposed in an appropriate solution, and the solution is crystallized. The solution can contain, for example, one or more polymers (e.g., polyethylene glycol (PEG)), one or more salts (e.g., potassium sulfate) and optionally one or more organic solvents. The crystals can be grown by various methods, such as, for example, sitting or hanging drop vapor diffusion. In general, crystallization can be performed at a temperature of from about 4° C. to 60° C. (e.g., from about 4° C. to about 45° C., such as at about 4° C., about 15° C., about 18° C., about 20° C., about 25° C., about 30° C., about 32° C., about 35° C., about 37° C.). Structural data describing a crystal of the mAb13.2 Fab fragment can be obtained, for example, by X-ray diffraction. X-ray diffraction data can be collected using a variety of means in order to obtain structural coordinates. Suitable X-ray sources include rotating anodes and synchrotron sources (e.g., Advanced Light Source (ALS), Berkeley, California; or Advanced Photon Source (APS), Argonne, Illinois). In certain embodiments, X-rays for generating diffraction data can have a wavelength of from about 0.5 Å to about 1.6 Å (e.g., about 0.7 Å, about 0.9 Å, about 1.0 Å, about 1.1 Å, about 1.3 Å, about 1.4 Å, about 1.5 Å, about 1.6 Å). Suitable X-ray detectors include area detectors and/or charge-couple devices (CCDs) can be used as the detector(s).

In general, a crystal of the mAb 13.2 Fab fragment can diffract X-rays to a resolution of about 3.5 Å or less (e.g., about 3.2 Å or less, about 3.0 Å or less, about 2.8 Å or less, about 2.5 Å or less, about 2.4 Å or less, about 2.3 Å or less, about 2.2 Å or less, about 2.1 Å or less, about 2.0 Å or less, about 1.9 Å or less, about 1.8 Å or less, about 1.7 Å or less, about 1.6 Å or less, about 1.5 Å or less, about 1.4 Å or less). In some embodiments, a crystal of the mAb13.2 Fab fragment can diffract X-rays to a resolution of from about 1.6 Å to about 2.5 Å (e.g., from about 1.8 Å to about 2.2 Å).

In certain embodiments, a crystal of the mAb13.2 Fab fragment can be orthorhombic with space group P2₁2₁2₁, and unit cell dimensions a=54.4, b=98.0, c=108.5, and α=β=γ=90° C.

In general, a complex including human IL-13 and the mAb13.2 Fab fragment can be prepared and crystallized as desired. In some embodiments, the process is as follows. Human IL-13 is expressed from a DNA plasmid. The expression can be driven by a promoter, such as an inducible promoter. Human IL-13 can be expressed as a fusion protein with a suitable tag (e.g., to facilitate isolation of human IL-13 from cells), such as a glutathione-S-transferase (GST), myc, HA, hexahistidine, or FLAG tag. A fusion protein can be cleaved at a protease site engineered into the fusion protein, such as at or near the site of fusion between the polypeptide and the tag. Human IL-13 can be mixed with the mAb13.2 Fab fragment prior to purification (e.g., prior to cleavage of a polypeptide tag), or human IL-13 can be mixed with the mAb13.2 Fab fragment after purification. In some embodiments, the mAb13.2 Fab fragment can be mixed with human IL-13 prior to purification and again following purification. In some embodiments, human IL-13 polypeptide and the mAb13.2 Fab fragment are combined in a solution for collecting spectral data for the complex, NMR data for the complex, or for growing a crystal of the complex. The solution can contain, for example, one or more salts (e.g., a potassium salt), one or more polymers (e.g., polyethylene glycol (PEG)), and/or one or more organic solvents. Crystals can be grown by various methods, such as, for example, sitting or hanging drop vapor diffusion. In general, crystallization can be performed at about 16° C. to 24° C. (e.g., about 17° C. to 23° C., or 18° C. to 21° C.).

Structural information for a crystal of a human IL-13/mAb13.2 Fab fragment complex can be obtained by X-ray diffraction. In general, a crystal of a human IL-13/mAb13.2 Fab fragment complex can diffract X-rays to a resolution of about 3.5 Å or less (e.g., about 3.2 Å or less, about 3.0 Å or less, about 2.8 Å or less, about 2.5 Å or less, about 2.4 Å or less, about 2.3 Å or less, about 2.2 Å or less, about 2.1 Å or less, about 2.0 Å or less, about 1.9 Å or less, about 1.8 Å or less, about 1.7 Å or less, about 1.6 Å or less, about 1.5 Å or less, about 1.4 Å or less). In some embodiments, a crystal of a human IL-13/mAb13.2 Fab fragment complex can diffract X-rays to a resolution of from about 1.6 Å to about 2.5 Å (e.g., from about 1.8 Å to about 2.2 Å).

In certain embodiments, a crystal of a human IL-13/mAb13.2 Fab fragment complex can be cubic with space group P2₁3, and unit cell dimensions a=b=c=125.3, and α=β=γ=90° C. The structure of the complex can be solved to a resolution of 1.8 Å.

In general, a complex including human IL-13, the mAb13.2 Fab fragment, and a human IL-13Rα1 polypeptide can be prepared and crystallized as desired. In some embodiments, the process is as follows. A human IL-13Rα1 polypeptide is expressed from a DNA plasmid in the yeast strain Pichia pastoris, such that the expressed polypeptide is glycosylated. Expression from the DNA plasmid can be driven by a promoter, such as an inducible promoter. The human IL-13Rα1 polypeptide can be expressed as a fusion protein with a suitable tag (e.g., to facilitate isolation of the human IL-13Rα1 polypeptide from cells), such as a glutathione-5-transferase (GST), myc, HA, hexahistidine, or FLAG tag. A fusion protein can be cleaved at a protease site engineered into the fusion protein, such as at or near the site of fusion between the polypeptide and the tag. The human IL-13Rα1 polypeptide can be mixed with human IL-13 to form a complex, and then the polypeptides of the complex can be deglycosylated by treatment with an enzyme such as endoglycosidase H. The mAb13.2 Fab fragment can be added to the deglycosylated complex to form a human IL-13Rα1 polypeptide/human IL-13/mAb13.2 Fab complex.

In some embodiments, the human IL-13Rα1, human IL-13, and mAb13.2 Fab fragment are combined in a solution for collecting spectral data for the complex, NMR data for the complex, or for growing a crystal of the complex. The solution can contain, for example, one or more salts (e.g., a potassium salt), one or more polymers (e.g., polyethylene glycol (PEG)), and/or one or more organic solvents. Crystals can be grown by various methods, such as, for example, sitting or hanging drop vapor diffusion. In general, crystallization can be performed at about 16° C. to 24° C. (e.g., about 17° C. to 23° C., or 18° C. to 21° C.).

Structural information for a crystal of a human IL-13Rα1 polypeptide/human IL-13/mAb13.2 Fab fragment complex can be obtained by X-ray diffraction. In general, a crystal of a human IL-13Rα1 polypeptide/human IL-13/mAb13.2 Fab fragment complex can diffract X-rays to a resolution of about 3.5 Å or less (e.g., about 3.2 Å or less, about 3.0 Å or less, about 2.8 Å or less, about 2.5 Å or less, about 2.4 Å or less, about 2.3 Å or less, about 2.2 Å or less, about 2.1 Å or less, about 2.0 Å or less, about 1.9 Å or less, about 1.8 Å or less, about 1.7 Å or less, about 1.6 Å or less, about 1.5 Å or less, about 1.4 Å or less). In some embodiments, a crystal of a human IL-13/mAb13.2 Fab fragment complex can diffract X-rays to a resolution of from about 1.6 Å to about 2.5 Å (e.g., from about 1.8 Å to about 2.2 Å).

In certain embodiments, a crystal of a human IL-13Rα 1 polypeptide/human IL-13/mAb13.2 Fab fragment complex can be cubic with space group 14, and unit cell dimensions a=b=164.9 Å, c=74.8 Å, and α=β=γ=90° C. The structure of the complex can be solved to a resolution of 2.2 Å.

X-ray diffraction data of a crystal of the mAb13.2 Fab fragment, human IL-13/mAb13.2 Fab fragment complex, or human IL-13Rα1 polypeptide/human IL-13/mAb13.2 Fab fragment complex can be used to obtain the structural coordinates of the atoms in the antibody or the complex. The structural coordinates are Cartesian coordinates that describe the location of atoms in three-dimensional space in relation to other atoms in the complex. As an example, the structural coordinates listed in Table 10 are the structural coordinates of a crystalline mAb13.2 Fab fragment. These structural coordinates describe the location of atoms of the mAb13.2 Fab fragment in relation to each other. As another example, the structural coordinates listed in Table 11 are the structural coordinates of a crystalline human IL-13/mAb13.2 Fab fragment complex. These structural coordinates describe the location of atoms of the human IL-13 in relation to each other, the location of atoms in the human IL-13 in relation to the atoms in the mAb13.2 Fab fragment, and the location of atoms in the mAb13.2 Fab fragment in relation to each other. As yet another example, the structural coordinates listed in Table 12 are the structural coordinates of a crystalline human IL-13Rα1 polypeptide/human IL-13/mAb13.2 Fab fragment complex. These structural coordinates describe the location of atoms of the IL-13Rα1 polypeptide in relation to each other, the location of atoms in the human IL-13Rα1 polypeptide in relation to the atoms in human IL-13, the location of atoms in human IL-13 in relation to each other, the location of atoms in human IL-13 in relation to the atoms in the mAb13.2 Fab fragment and the location of atoms in the mAb13.2 Fab fragment in relation to each other.

The structural coordinates of a crystal can be modified by mathematical manipulation, such as by inversion or integer additions or subtractions. As such, structural coordinates are relative coordinates. As an example, structural coordinates describing the location of atoms in the mAb13.2 Fab fragment are not specifically limited by the actual x, y, and z coordinates of Table 10. As another example, structural coordinates describing the location of atoms in the human IL-13 bound to the mAb13.2 Fab fragment are not specifically limited by the actual x, y, and z coordinates of Table 11. As yet another example, structural coordinates describing the location of atoms in the human IL-13 bound to both the mAb13.2 Fab fragment and the human IL-13Rα1 polypeptide are not specifically limited by the actual x, y, and z coordinates of Table 12.

The structural coordinates of the mAb13.2 Fab fragment or human IL-13/mAb13.2 Fab fragment complex or human IL-Rα1 polypeptide/human IL-13/mAb13.2 Fab fragment complex can be used to derive a representation (e.g., a two dimensional representation or three dimensional representation) of the mAb13.2 Fab fragment, a fragment of the mAb13.2 Fab fragment, human IL-13, a fragment of human IL-13, the human IL-13Rα1 polypeptide, a fragment of the IL-13Rα1 polypeptide, the human IL-13/mAb13.2 Fab fragment complex or human IL-Rα1 polypeptide/human IL-13/mAb 13.2 Fab fragment complex, or a fragment of either complex. Such a representation can be useful for a number of applications, including, for example, the visualization, identification and characterization of an active site of the polypeptide. In certain embodiments, a three-dimensional representation can include the structural coordinates of the mAb13.2 fragment according to Table 10±a root mean square deviation from the alpha carbon atoms of amino acids of about 1.5 Å or less (e.g., about 1.0 Å or less, or about 0.5 Å or less). In other embodiments, a three-dimensional representation can include the structural coordinates of a human IL-13/mAb13.2 Fab fragment complex according to Table 11±a root mean square deviation from the alpha carbon atoms of amino acids of not more than about 1.5 Å (e.g., not more than about 1.0 Å, not more than about 0.5 Å or less). In yet other embodiments, a three-dimensional representation can include the structural coordinates of a human IL-13Rα1 polypeptide/human IL-13/mAb13.2 Fab fragment complex according to Table 12±a root mean square deviation from the alpha carbon atoms of amino acids of not more than about 1.5 Å (e.g., not more than about 1.0 Å, not more than about 0.5 Å or less). Root mean square deviation (rms deviation, or rmsd) is the square root of the arithmetic mean of the squares of the deviations from the mean, and is a way of expressing deviation or variation from structural coordinates. Conservative substitutions of amino acids can result in a molecular representation having structural coordinates within the stated root mean square deviation. For example, two molecular models of polypeptides that differ from one another by conservative amino acid substitutions can have coordinates of backbone atoms within a stated rms deviation, such as less than about 1.5 Å (e.g., less than about about 1.0 Å, less than about 0.5 Å). Backbone atoms of a polypeptide include the alpha carbon (C_{α} or CA) atoms, carbonyl carbon (C) atoms, and amide nitrogen (N) atoms.

Various software programs allow for the graphical representation of a set of structural coordinates to obtain a representation of a molecule or molecular complex, such as the mAb13.2 Fab fragment or the human IL-13/mAb 13.2 Fab fragment complex or the human IL-13Rα1 polypeptide/human IL-13/mAb13.2 Fab fragment complex. In general, such a representation should accurately reflect (relatively and/or absolutely) structural coordinates, or information derived from structural coordinates, such as distances or angles between features. The representation can be a two-dimensional figure, such as a stereoscopic two-dimensional figure, or an interactive two-dimensional display (e.g., a computer display that can display different faces of the molecule or molecular complex), or an interactive stereoscopic two-dimensional display. An interactive two-dimensional display can be, for example, a computer display that can be rotated to show different faces of a polypeptide, a fragment of a polypeptide, a complex and/or a fragment of a complex. In some embodiments, the representation is a three-dimensional representation. As an example, a three-dimensional model can be a physical model of a molecular structure (e.g., a ball-and-stick model). As another example, a three dimensional representation can be a graphical representation of a molecular structure (e.g., a drawing or a figure presented on a computer display). A two-dimensional graphical representation (e.g., a drawing) can correspond to a three-dimensional representation when the two-dimensional representation reflects three-dimensional information, for example, through the use of perspective, shading, or the obstruction of features more distant from the viewer by features closer to the viewer. In some embodiments, a representation can be modeled at more than one level. As an example, when the three-dimensional representation includes a polypeptide, such as human IL-13 bound to the mAb13.2 Fab fragment, the polypeptide can be represented at one or more different levels of structure, such as primary structure (amino acid sequence), secondary structure (e.g., α-helices and β-sheets), tertiary structure (overall fold), and quaternary structure (oligomerization state). The heavy and light chain polypeptides of the mAb13.2 Fab fragment can also be represented at the one or more different structural levels. A representation can include different levels of detail. For example, the representation can include the relative locations of secondary structural features of a protein without specifying the positions of atoms. A more detailed representation could, for example, include the positions of atoms.

In some embodiments, a representation can include information in addition to the structural coordinates of the atoms in the mAb13.2 Fab fragment, the human IL-13/mAb13.2 Fab fragment complex, or the human IL-13Rα1 polypeptide/human IL-13/mAb13.2 Fab fragment complex. For example, a representation can provide information regarding the shape of a solvent accessible surface, the van der Waals radii of the atoms of the model, and the van der Waals radius of a solvent (e.g., water). Other features that can be derived from a representation include, for example, electrostatic potential, the location of voids or pockets within a macromolecular structure, and the location of hydrogen bonds and salt bridges.

An agent that interacts with the mAb13.2 Fab fragment, human IL-13, or the human IL-13Rα1 polypeptide can be identified or designed by a method that includes using a representation of the mAb13.2 Fab fragment, a human IL-13, a human IL-13Rα1 polypeptide, a human IL-13/mAb13.2 Fab fragment complex, or a human IL-13-Rα1 polypeptide/human IL-13/mAb13.2 Fab fragment complex. Exemplary types of representations include the representations discussed above. In some embodiments, the representation can be of an analog polypeptide, polypeptide fragment, complex or fragment of a complex. A candidate agent that interacts with the representation can be designed or identified by performing computer fitting analysis of the candidate agent with the representation. In general, an agent is a molecule. Examples of agents include polypeptides, nucleic acids (including DNA or RNA), or small molecules (e.g., small organic molecules). An agent can be a ligand, and can act, for example, as an agonist or antagonist. An agent that interacts with a polypeptide (e.g., human IL-13, human IL-13Rα1 polypeptide) can interact transiently or stably with the polypeptide. The interaction can be mediated by any of the forces noted herein, including, for example, hydrogen bonding, electrostatic forces, hydrophobic interactions, and van der Waals interactions.

As noted above, X-ray crystallography can be used to obtain structural coordinates of an mAb13.2 Fab fragment, a human IL-13/mAb13.2 Fab fragment complex, or a human IL-13Rα1 polypeptide/human IL-13/mAb13.2 Fab fragment complex. However, such structural coordinates can be obtained using other techniques including NMR techniques. Additional structural information can be obtained from spectral techniques (e.g., optical rotary dispersion (ORD), circular dichroism (CD)), homology modeling, and computational methods such as those that include data from molecular mechanics or from dynamics assays).

In some embodiments, the X-ray diffraction data can be used to construct an electron density map of the mAb13.2 Fab fragment, the human IL-13/mAb13.2 Fab fragment complex, or the human IL-13Rα1 polypeptide/human IL-13/mAb13.2 Fab fragment complex. The electron density map can be used to derive a representation (e.g., a two dimensional representation or a three dimensional representation) of the mAb13.2 Fab fragment, a fragment of the mAb13.2 Fab fragment, human IL-13 or a fragment of human IL-13, the human IL-13Rα1 polypeptide or a fragment of the human IL-13Rα1 polypeptide, the human IL-13/mAb13.2 Fab fragment complex, the human IL-13Rα1 polypeptide/human IL-13/mAb 13.2 Fab fragment complex, or a fragment of either complex. Creation of an electron density map typically involves using information regarding the phase of the X-ray scatter. Phase information can be extracted, for example, either from the diffraction data or from supplementing diffraction experiments to complete the construction of the electron density map. Methods for calculating phase from X-ray diffraction data include, without limitation, multiwavelength anomalous dispersion (MAD), multiple isomorphous replacement (MIR), multiple isomorphous replacement with anomalous scattering (MIRAS), single isomorphous replacement with anomalous scattering (SIRAS), reciprocal space solvent flattening, molecular replacement, or a combination thereof. These methods generate phase information by making isomorphous structural modifications to the native protein, such as by including a heavy atom or changing the scattering strength of a heavy atom already present, and then measuring the diffraction amplitudes for the native protein and each of the modified cases. If the position of the additional heavy atom or the change in its scattering strength is known, then the phase of each diffracted X-ray can be determined by solving a set of simultaneous phase equations. The location of heavy atom sites can be identified using a computer program, such as SHELXS (Sheldrick, Institut Anorg. Chemie, Göttingen, Germany), and diffraction data can be processed using computer programs such as MOSFLM, SCALA, SOLOMON, and SHARP (“The CCP4 Suite: Programs for Protein Crystallography,” Acta Crystallogr. Sect. D, 54:905-921, 1997; deLa Fortelle and Brigogne, Meth. Enzym. 276:472-494, 1997). Upon determination of the phase, an electron density map of the complex can be constructed.

The electron density map can be used to derive a representation of a polypeptide, a complex, or a fragment of a polypeptide or complex by aligning a three-dimensional model of a polypeptide or complex (e.g., a complex containing a polypeptide bound to an antibody) with the electron density map. The alignment process results in a comparative model that shows the degree to which the calculated electron density map varies from the model of the previously known polypeptide or the previously known complex. The comparative model is then refined over one or more cycles (e.g., two cycles, three cycles, four cycles, five cycles, six cycles, seven cycles, eight cycles, nine cycles, ten cycles) to generate a better fit with the electron density map. A software program such as CNS (Brunger et al., Acta Crystallogr. D54:905-921, 1998) can be used to refine the model. The quality of fit in the comparative model can be measured by, for example, an R_{work} or R_{free} value. A smaller value of R_{work} or R_{free} generally indicates a better fit. Misalignments in the comparative model can be adjusted to provide a modified comparative model and a lower R_{work} or R_{free} value. The adjustments can be based on information relating to human IL-13, human IL-13Rα 1, the mAb13.2 Fab fragment, the previously known polypeptide and/or the previously known complex. Such information includes, for example, estimated helical or beta sheet content, hydrophobic and hydrophilic domains, and protein folding patterns, which can be derived, for example, from amino acid sequence, homology modeling, and spectral data. As an example, in embodiments in which a model of a previously known complex of a polypeptide bound to a ligand is used, an adjustment can include replacing the ligand in the previously known complex with the mAb13.2 fragment. As another example, in certain embodiments, an adjustment can include replacing an amino acid in the previously known polypeptide with the amino acid in the corresponding site of human IL-13. When adjustments to the modified comparative model satisfy a best fit to the electron density map, the resulting model is that which is determined to describe the antibody or polypeptide or complex from which the X-ray data was derived (e.g., the human IL-13/mAb13.2 Fab fragment complex). Methods of such processes are disclosed, for example, in Carter and Sweet, eds., “Macromolecular Crystallography” in Methods in Enzymology, Vol. 277, Part B, New York: Academic Press, 1997, and articles therein, e.g., Jones and Kjeldgaard, “Electron-Density Map Interpretation,” p. 173, and Kleywegt and Jones, “Model Building and Refinement Practice,” p. 208.

In some embodiments, a representation of the mAb13.2 Fab fragment can be derived by aligning a previously determined structural model of a different (but similar) antibody Fab fragment (e.g., a 2E8 Fab antibody fragment, Protein Databank Identification No. 12E8) with the electron density map of the mAb13.2 Fab fragment derived from X-ray diffraction data. A representation of a human IL-13/mAb13.2 Fab fragment complex can subsequently be derived by aligning the previously determined structural model of the mAb13.2 Fab fragment with the electron density map of the complex. A representation of a human IL-13Rα1 polypeptide/human IL-13/mAb13.2 Fab fragment complex can subsequently be derived by aligning the previously determined structural model of the human IL-13/mAb13.2 Fab fragment complex with the electron density map of the human IL-13Rα1 polypeptide/human IL-13/mAb13.2 Fab fragment complex.

A machine, such as a computer, can be programmed in memory with the structural coordinates of the mAb13.2 Fab fragment, a human IL-13/mAb13.2 Fab fragment complex, or a human IL-13Rα1 polypeptide/human IL-13/mAb13.2 Fab fragment complex together with a program capable of generating a three-dimensional graphical representation of the structural coordinates on a display connected to the machine. Alternatively or additionally, a software system can be designed and/or utilized to accept and store the structural coordinates. The software system can be capable of generating a graphical representation of the structural coordinates. The software system can also be capable of accessing external databases to identify compounds (e.g., polypeptides) with similar structural features as human IL-13 or human IL-13Rα 1, and/or to identify one or more candidate agents with characteristics that may render the candidate agent(s) likely to interact with human IL-13 or human IL-13Rα1. The software system can also be capable of accessing external databases to identify compounds that interact with human IL-13 or human IL-13Rα1 by virtue of the knowledge of the structure of the mAb13.2 Fab fragment, or human IL-13Rα1 polypeptide, and its interaction with human IL-13.

A machine having a memory containing structure data or a software system containing such data can aid in the rational design or selection of IL-13 ligands, such as agonists or antagonists. For example, such a machine or software system can aid in the evaluation of the ability of an agent to associate with human IL-13, can aid in the modeling of compounds or proteins related by structural or sequence homology to human IL-13, or can aid in the evaluation of the ability of an agent to interfere with the bioactivity of human IL-13. A bioactivity of human IL-13 can be any effect that the polypeptide elicits on or in a cell or tissue in vivo or in vitro. Exemplary bioactivities of human IL-13 are described herein, such as in Examples 1 and 2.

A machine having a memory containing structure data or a software system containing such data can aid in the rational design or selection of IL-13Rα1 ligands, such as agonists or antagonists. For example, such a machine or software system can aid in the evaluation of the ability of an agent to associate with a human IL-13Rα1 polypeptide, can aid in the modeling of compounds or proteins related by structural or sequence homology to a human IL-13Rα1 polypeptide, or can aid in the evaluation of the ability of an agent to interfere with the bioactivity of a human IL-13Rα1 polypeptide. A bioactivity of a human IL-13Rα1 polypeptide can be any affect that the polypeptide elicits on or in a cell or tissue in vivo or in vitro. Exemplary bioactivities of human IL-13Rα1 are described herein, such as in Example 3.

The machine can produce a representation (e.g., a two dimensional representation or a three dimensional representation) of the mAb13.2 Fab fragment or a fragment of the mAb13.2 Fab fragment, human IL-13 or a fragment of human IL-13, a human IL-13Rα1 polypeptide or a fragment of a human IL-13Rα1 polypeptide, a human IL-13/mAb13.2 Fab fragment complex, a human IL-13Rα1 polypeptide/human IL-13/mAb13.2 fab fragment complex, or a fragment of either complex. A software system, for example, can cause the machine to produce such information. The machine can include a machine-readable data storage medium including a data storage material encoded with machine-readable data. The machine-readable data can include structural coordinates of atoms of the mAb13.2 Fab fragment or atoms of a fragment of the mAb13.2 Fab fragment, atoms of human IL-13 or atoms of a fragment of human IL-13, atoms of a human IL-13/mAb 13.2 Fab fragment complex, atoms of a human IL-13Rα1 polypeptide/human IL-13/mAb13.2 fab fragment complex, or atoms of either complex. Machine-readable storage media including data storage material can include conventional computer hard drives, floppy disks, DAT tape, CD-ROM, DVD, and other magnetic, magneto-optical, optical, and other media which may be adapted for use with a computer. The machine can also have a working memory for storing instructions for processing the machine-readable data, as well as a central processing unit (CPU) coupled to the working memory and to the machine-readable data storage medium for the purpose of processing the machine-readable data into the desired three-dimensional representation. Finally, a display can be connected to the CPU so that the three-dimensional representation may be visualized by the user. Accordingly, when used with a machine programmed with instructions for using the data (e.g., a computer loaded with one or more programs of the sort described herein) the machine is capable of displaying a graphical representation (e.g., a two dimensional graphical representation, a three-dimensional graphical representation) of any of the polypeptides, polypeptide fragments, complexes, or complex fragments described herein.

A display (e.g., a computer display) can show a representation of the mAb13.2 Fab fragment or a fragment of the mAb 13.2 Fab fragment, human IL-13 or a fragment of human IL-13, a human IL-13Rα1 polypeptide or a fragment of a human IL-13Rα1 polypeptide, a human IL-13/mAb13.2 Fab fragment complex, a human IL-13Rα1 polypeptide/human IL-13/mAb 13.2 fab fragment complex, or a fragment of either complex. The representation can also include an agent bound to human IL-13 or the human IL-13Rα1 polypeptide, or the user can superimpose a three-dimensional model of an agent on the representation of human IL-13 or the human IL-13Rα1 polypeptide. The agent can be an agonist (e.g., a candidate agonist) of human IL-13 or human IL-13Rα1, or an antagonist (e.g., a candidate antagonist) of human IL-13 or human IL-13Rα1. In some embodiments, the agent can be a known compound or fragment of a compound. In certain embodiments, the agent can be a previously unknown compound, or a fragment of a previously unknown compound.

The user can inspect the resulting representation. A representation of the mAb13.2 Fab fragment or fragment of the mAb13.2 Fab fragment, human IL-13 or fragment of the human IL-13, the human IL-13Rα1 polypeptide or fragment of the human IL-13Rα1 polypeptide, the human IL-13/mAb13.2 Fab fragment complex, the human IL-13Rα1 polypeptide/human IL-13/mAb13.2 fab fragment complex, or the fragment of either complex can be generated, for example, by altering a previously existing representation of such polypeptides and polypeptide complexes. For example, there can be a preferred distance, or range of distances, between atoms of the antibody and atoms of the human IL-13 when considering a new representation of a complex or fragment of a complex. In another example, there can be a preferred distance, or range of distances, between atoms of the human IL-13 and the human IL-13Rα 1 polypeptide when considering a new representation of a complex or fragment of a complex. Distances longer than a preferred distance may be associated with a weak interaction between the agent and active site (e.g., the site of IL-13 receptor binding (such as to an IL-13Rα1 receptor polypeptide or an IL-4 receptor polypeptide) on the IL-13 polypeptide). Distances shorter than a preferred distance may be associated with repulsive forces that can weaken the interaction between the agent and the polypeptide. A steric clash can occur when distances between atoms are too short. A steric clash occurs when the locations of two atoms are unreasonably close together, for example, when two atoms are separated by a distance less than the sum of their van der Waals radii. If a steric clash exists, the user can adjust the position of the agent relative to the human IL-13 (e.g., a rigid body translation or rotation of the agent), until the steric clash is relieved. The user can adjust the conformation of the agent or of the human IL-13 in the vicinity of the agent in order to relieve a steric clash. Steric clashes can also be removed by altering the structure of the agent, for example, by changing a “bulky group,” such as an aromatic ring, to a smaller group, such as to a methyl or hydroxyl group, or by changing a rigid group to a flexible group that can accommodate a conformation that does not produce a steric clash. Electrostatic forces can also influence an interaction between an agent and a polypeptide (such as the part of the polypeptide that interacts with a receptor polypeptide, e.g., a human IL-13Rα1 polypeptide or a human IL-4R polypeptide). For example, electrostatic properties can be associated with repulsive forces that can weaken the interaction between the agent and the IL-13 polypeptide. Altering the charge of the agent, e.g., by replacing a positively charged group with a neutral group can relieve electrostatic repulsion. Similar processes can be performed to design an agent that interacts with a human IL-13Rα1 polypeptide, such as in the vicinity of interaction between the human IL-13Rα1 polypeptide and human IL-13.

Forces that influence binding strength between the mAb13.2 Fab fragment and human IL-13 can be evaluated in the polypeptide/agent model. Likewise, forces that influence binding strength between human IL-13 and the human IL-13Rα1 polypeptide can be evaluated in the polypeptide/agent model. These can include, for example, hydrogen bonding, electrostatic forces, hydrophobic interactions, van der Waals interactions, dipole-dipole interactions, π-stacking forces, and anion-π interactions. The user can evaluate these forces visually, for example by noting a hydrogen bond donor/acceptor pair arranged with a distance and angle suitable for a hydrogen bond. Based on the evaluation, the user can alter the model to find a more favorable interaction between the human IL-13, or human IL-13Rα1 polypeptide, and the agent. Altering the model can include changing the three-dimensional structure of the polypeptide without altering its chemical structure, for example by altering the conformation of amino acid side chains or backbone dihedral angles. Altering the model can include altering the position or conformation of the agent, as described above. Altering the model can also include altering the chemical structure of the agent, for example by substituting, adding, or removing groups. For example, if a hydrogen bond donor on the human IL-13 is located near a hydrogen bond donor on the agent, the user can replace the hydrogen bond donor on the agent with a hydrogen bond acceptor.

The relative locations of the agent and the human IL-13, or their conformations, can be adjusted to find an optimized binding geometry for a particular agent to the IL-13 polypeptide. Likewise, the relative locations of the agent and the human IL-13Rα1 polypeptide can be adjusted to find an optimized binding geometry for a particular agent to the human IL-13Rα1 polypeptide. An optimized binding geometry is characterized by, for example, favorable hydrogen bond distances and angles, maximal electrostatic attractions, minimal electrostatic repulsions, the sequestration of hydrophobic moieties away from an aqueous environment, and the absence of steric clashes. The optimized geometry can have the lowest calculated energy of a family of possible geometries for a human IL-13/antibody complex, or a human IL-13/receptor complex. An optimized geometry can be determined, for example, through molecular mechanics or molecular dynamics calculations.

A series of representations of human IL-13 bound to different agents can be generated. Likewise, a series of representations of a human IL-13Rα1 polypeptide bound to different agents can be generated. A score can be calculated for each representation. The score can describe, for example, an expected strength of interaction between human IL-13 and the agent. The score can reflect one of the factors described above that influence binding strength. The score can be an aggregate score that reflects more than one of the factors. The different agents can be ranked according to their scores.

Steps in the design of the agent can be carried out in an automated fashion by a machine (e.g., a computer). For example, a representation of human IL-13, or a human IL-13Rα1 polypeptide can be programmed in the machine, along with representations of candidate agents. The machine can find an optimized binding geometry for each of the candidate agents to the site of receptor binding, and calculate a score to determine which of the agents in the series is likely to interact most strongly with human IL-13, or the human IL-13Rα1 polypeptide.

A software system can be designed and/or implemented to facilitate these steps. Software systems (e.g., computer programs) used to generate representations or perform the necessary fitting analyses include, but are not limited to: MCSS, Ludi, QUANTA, Insight II, Cerius2, CHARMm, and Modeler from Accelrys, Inc. (San Diego, Calif.); SYBYL, Unity, FleXX, and LEAPFROG from TRIPOS, Inc. (St. Louis, Mo.); AUTODOCK (Scripps Research Institute, La Jolla, Calif.), GRID (Oxford University, Oxford, UK); DOCK (University of California, San Francisco, Calif.); and Flo⁺ and Flo99 (Thistlesoft, Morris Township, N.J.). Other useful programs include ROCS, ZAP, FRED, Vida, and Szybki from Openeye Scientific Software (Santa Fe, N. Mex.); Maestro, Macromodel, and Glide from Schrodinger, LLC (Portland, Oreg.); MOE (Chemical Computing Group, Montreal, Quebec), Allegrow (Boston De Novo, Boston, Mass.), CNS (Brunger, et al., Acta Crystall. Sect. D 54:905-921, 1997) and GOLD (Jones et al., J. Mol. Biol. 245:43-53, 1995. The structural coordinates can also be used to visualize the three-dimensional structure of human IL-13 using MOLSCRIPT, RASTER3D, or PYMOL (Kraulis, J. Appl. Crystallogr. 24: 946-950, 1991; Bacon and Anderson, J. Mol. Graph. 6: 219-220, 1998; DeLano, The PYMOL Molecular Graphics System (2002) DeLano Scientific, San Carlos, Calif.).

The agent can, for example, be selected by screening an appropriate database, can be designed de novo by analyzing the steric configurations and charge potentials of an unbound human IL-13, or unbound human IL-13Rα1 polypeptide, in conjunction with the appropriate software systems, and/or can be designed using characteristics of known cytokine ligands. The agent can be tested for an ability to block binding of IL-13 to an IL-4R polypeptide, such as IL-4Rα, or an IL-Rα1 polypeptide. An agent can be designed for binding to human IL-13 or to the human IL-13Rα1 polypeptide. The method can be used to design or select agonists or antagonists of human IL-13 or a human IL-Rα1 polypeptide. A software system can be designed and/or implemented to facilitate database searching, and/or agent selection and design.

Once an agent has been designed or identified, it can be obtained or synthesized and further evaluated for its affect on human IL-13 activity or on human IL-13Rα1 activity. The agent can be evaluated by contacting it with human IL-13 and assaying IL-13 bioactivity, or by contacting it with a human IL-13Rα1 polypeptide and assaying IL-13Rα1 bioactivity. A method for evaluating the agent can include an activity assay performed in vitro or in vivo. An activity assay can be a cell-based assay, for example. Depending upon the action of the agent on human IL-13 or the human IL-13Rα1 polypeptide, the agent can act either as an agonist or antagonist of human IL-13 or IL-13Rα1 activity. An agonist will cause human IL-13 or human IL-13Rα1 polypeptide to have the same or similar activity, and an antagonist will inhibit a normal function of human IL-13 or the human IL-13Rα1 polypeptide. An agent can be contacted with the human IL-13 in the presence of an anti-IL-13 antibody (e.g., mAb13.2 or mAb13.2 Fab) or a human IL-13 receptor (e.g., an IL-4R polypeptide, such as a human IL-4Rα polypeptide, or an IL-13R polypeptide, such as a human IL-13Rα1 polypeptide) to determine whether or not the agent inhibits binding of the antibody or the receptor to the human IL-13 polypeptide. In some embodiments, the agent will inhibit binding of one kind of receptor to human IL-13, but will not inhibit binding of another kind of receptor. For example, an agent can inhibit binding of a human IL-13 polypeptide to a human IL-4R polypeptide (e.g., the IL-4Rα chain), but not a human IL-13Rα1 polypeptide. Likewise, a different agent can inhibit binding of human IL-13 to an IL-13Rα1 polypeptide but not to a human IL-4R polypeptide. In another embodiment, the agent will inhibit binding of the IL-13 polypeptide to a human IL 4R polypeptide (e.g., the IL-4Rα chain) and a human IL-13Rα1 polypeptide. A crystal containing human IL-13 bound to the identified agent can be grown and the structure determined by X-ray crystallography. A second agent can be designed or identified based on the interaction of the first agent with human IL-13. Various molecular analysis and rational drug design techniques are further disclosed in, for example, U.S. Pat. Nos. 5,834,228, 5,939,528 and 5,856,116, as well as in PCT Application No. PCT/US98/16879, published as WO 99/09148.

While certain embodiments have been described, other embodiments are also contemplated.

As an example, while embodiments involving human IL-13, the mAb 13.2 Fab fragment, and a human IL-13Rα1 polypeptide have been described, more generally, any IL-13 polypeptide, any IL-13Rα 1 polypeptide, and/or any anti-IL-13 antibody can be used.

As an example, while embodiments have been described that involve human IL-13 and a human IL-13Rα 1 polypeptide, more generally any IL-13 polypeptide and any IL-13Rα1 polypeptide can be used. For example, an IL-13 polypeptide or an IL-13Rα1 polypeptide can originate from a nonmammalian or mammalian species. Exemplary nonhuman mammals include, a nonhuman primate (such as a monkey or ape), a mouse, rat, goat, cow, bull, pig, horse, sheep, wild boar, sea otter, cat, or dog. Exemplary nonmammalian species include chicken, turkey, shrimp, alligator, or fish.

Further, an IL-13 polypeptide or an IL-13Rα1 polypeptide can generally be a full-length, mature polypeptide, including the full-length amino acid sequence of any isoform or processed form of an IL-13 polypeptide or IL-13Rα1 polypeptide. An isoform is any of several multiple forms of a protein that differ in their primary structure. Full-length IL-13 can be referred to as the precursor form of the protein. Full-length IL-13 has a signal peptide cleavage site. The IL-13 polypeptide can be the processed polypeptide, such as following cleavage of the signal peptide.

A human IL-13 polypeptide typically has at least one active site for interacting with a receptor polypeptide (e.g., an IL-4R polypeptide, an IL-13α1 polypeptide). An IL-13 polypeptide can include three active sites for interacting with two different receptor polypeptides. An anti-IL-13 antibody can be capable of binding to at least one of the active sites. In general, an active site can include a site of receptor polypeptide binding, or a site of phosphorylation, glycosylation, alkylation, acylation, or other covalent modification. An active site can include accessory binding sites adjacent or proximal to the actual site of binding that may affect activity upon interaction with the ligand. An active site of a human IL-13 polypeptide can include amino acids of SEQ ID NO:4. For example, an active site of a human IL-13 polypeptide can include one or more of amino acids Ser7, Thr8, Ala9, Glu12, Leu48, Glu49, Ile52, Asn53, Arg65, Ser68, Gly69, Phe70, Cys71, Pro72, His73, Lys74, and Arg86 as defined by the amino acid sequence of SEQ ID NO:4 (FIG. 2B). In some embodiments, an agent can interact to within about 2.0A or less (e.g., about 1.5A or less, about 1.0 Å or less) of one or more amino acids Glu49, Asn53, Gly69, Pro72, His73, Lys74, and Arg86 of IL-13, as defined by the amino acid sequence of SEQ ID NO:4. In one alternative, an active site of a human IL-13 polypeptide can include one or more of amino acids Arg11, Glu12, Leu13, Ile14, Glu15, Lys104, Lys105, Leu106, Phe107, and Arg108 as defined by the amino acid sequence of SEQ ID NO:4. In another alternative, an active site of a human IL-13 polypeptide can include one or more of amino acids Thr88, Lys89, Ile90, and Glu91 as defined by the amino acid sequence of SEQ ID NO:4. A human IL-13 polypeptide can include one, two, or all three of the active sites described above.

A human IL-13Rα1 polypeptide typically has at least one active site for interacting with a polypeptide ligand (e.g., a human IL-13 polypeptide). An anti-IL-13Rα1 antibody can be capable of binding to at least one of the active sites. In general, an active site can include a site of polypeptide ligand binding, or a site of phosphorylation, glycosylation, alkylation, acylation, or other covalent modification. An active site can include accessory binding sites adjacent or proximal to the actual site of binding that may affect activity upon interaction with the ligand. An active site of a human IL-13Rα1 polypeptide can include amino acids of SEQ ID NO:12. For example, an active site of a human IL-13Rα1 polypeptide can include one or more of amino acid residues Ile254, Ser255, Arg256, Lys318, Cys320, and Tyr321 as defined by the amino acid sequence of SEQ ID NO:12. In one alternative, an active site of a human IL-13Rα1 polypeptide can include one or more of amino acid residues Lys76, Lys77, Ile78, and Ala79 as defined by the amino acid sequence of SEQ ID NO: 12. A human IL-13Rα1 polypeptide can include one or both of these active sites.

The numbering of the amino acids of a human IL-13 polypeptide, a human IL-13Rα1 polypeptide, and the heavy and light chains of an anti-IL-13 antibody, such as mAb13.2 Fab, may be different than that set forth here, and may contain certain conservative amino acid substitutions, additions or deletions that yield the same three-dimensional structure as those defined by Table 10, +an rmsd for backbone atoms of less than 1.5 Å, or by Table 11, ±an rmsd for backbone atoms of less than 1.5 Å, or by Table 12, ±an rmsd for backbone atoms of less than 1.5 Å. For example, the numbering of a human IL-13 processed polypeptide may be different than that set forth in FIG. 2B, and the sequence of the IL-13 may contain conservative amino acid substitutions but yield the same structure as that defined by the coordinates of Table 11 and illustrated in FIG. 3 or the same structure as that defined by the coordinates of Table 12 and illustrated in FIGS. 15,16 and 17. Corresponding amino acids and conservative substitutions in other isoforms or analogs are easily identified by visual inspection of the relevant amino acid sequences or by using commercially available homology software programs (e.g., MODELLAR, MSI, San Diego, Calif.).

An analog is a polypeptide having conservative amino acid substitutions. Conservative substitutions are amino acid substitutions that are functionally or structurally equivalent to the substituted amino acid. A conservative substitution can include switching one amino acid for another with similar polarity, or steric arrangement, or belonging to the same class (e.g., hydrophobic, acidic or basic) as the substituted amino acid. Conservative substitutions include substitutions having an inconsequential effect on the three-dimensional structure of an anti-IL-13 antibody or a human IL-13 polypeptide/anti-IL-13 antibody complex or a human IL-13Rα1 polypeptide/human IL-13 polypeptide/anti-IL-13 antibody complex with respect to identification and design of agents that interact with the polypeptide (e.g., an IL-13 polypeptide, an IL-13Rα1 polypeptide), as well as for molecular replacement analyses and/or for homology modeling.

While examples have been described in which an anti-IL-13 antibody is derived from a mouse, more generally any anti-IL-13 antibody can be used. For example, an anti-IL-13 antibody can originate from a human, mouse, rat, hamster, rabbit, goat, horse, or chicken.

As another example, while embodiments have been described in which an anti-IL-13 antibody is generated by a certain method, other methods may also be used. For example, an anti-IL-13 antibody can be generated by first preparing polyclonal antisera by immunization of female BALB/c mice with recombinant or native human IL-13. Sera can be screened for binding to human IL-13 by an assay such as ELISA. Splenocytes from a mouse demonstrating high serum antibody titers can be fused with a myeloma cell line, such as the P3X63_AG8.653 myeloma cell line (ATCC, Manassas, Va.), and plated in selective media. Fusions can be isolated following multiple rounds of subcloning by limiting dilution and the fusions can be screened for the production of antibodies that have a binding affinity to human IL-13. An anti-IL-13 antibody can be polyclonal or monoclonal. An antibody that binds IL-13 can be a fragment of an antibody, such as a Fab fragment.

In general, intact antibodies, also known as immunoglobulins, are tetrameric glycosylated proteins composed of two light (L) chains of approximately 25 kDa each and two heavy (H) chains of approximately 50 kDa each. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated as CH1. The VH and VL domain consist of four regions of relatively conserved sequence called framework regions, which form a scaffold for three regions of hypervariable sequence (complementarity determining regions, CDRs). The CDRs contain most of the residues responsible for specific interactions with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3 (see Table 4, for example). The subunit structures and three-dimensional configurations of different classes of immunoglobulins are well known in the art. For a review of antibody structure, see Antibodies: A Laboratory Manual, Cold Spring Harbor Laboratory, eds. Harlow et al. (1988). The smallest antigen-binding fragment is the Fv (Fragment variable), which consists of the VH and VL domains. The Fab (fragment antigen binding) fragment consists of the VH-CH 1 and VL-CL domains covalently linked by a disulfide bond between the constant regions.

Accordingly, in one aspect, this application features an antibody or an antigen-binding fragment thereof, that binds to and/or neutralizes, IL-13. The antibody or fragment thereof can also be a human, humanized, chimeric, or in vitro-generated antibody. In one embodiment, the anti-IL-13 antibody or fragment thereof is a humanized antibody. The antibody includes one or more CDRs that has a backbone conformation of a CDR described in Table 10±a root mean square deviation (RMSD) of not more than 1.5, 1.2, 1.1, or 1.0 Angstroms, Table 11±an RMSD of not more than 1.5, 1.2, 1.1, or 1.0 Angstroms, or Table 12±an RMSD of not more than 1.5, 1.2, 1.1, or 1.0 Angstroms. For example, one, two, or three of the CDRs of the light chain variable domain (e.g., particularly in CDR1, or in at least two CDRs, e.g., CDR1 and CDR3, CDR1 and CDR2, or in all three CDRs) have an RMSD of not more than 1.5, 1.2, 1.1, or 1.0 Angstroms, relative to those structures. In one embodiment, the antibody or antigen binding fragment thereof includes a variable domain that, as a whole, has a backbone conformation of a CDR described in Table 10±a root mean square deviation (RMSD) of not more than 1.5, 1.2, 1.1, or 1.0 Angstroms, Table 11±an RMSD of not more than 1.5, 1.2, 1.1, or 1.0 Angstroms, or Table 12±an RMSD of not more than 1.5, 1.2, 1.1, or 1.0 Angstroms. The variable domain can also be at least at least 70%, 80%, 85%, 87%, 90%, 92%, 93%, 95%, 96%, 97%, 98%, or 99% identical to an antibody described herein, e.g., in the CDR region and/or framework regions. The antibody can be used, e.g., in a method of treatment described herein.

Anti-IL-13 antibodies are disclosed, for example, in U.S. Provisional Patent Application No. 60/578,473, filed Jun. 9, 2004, U.S. Provisional Patent Application No. 60/581,375 filed Jun. 22, 2004, and U.S. patent application Ser. No. ______ [Attorney Docket: AM101493] (Kasaian et al.), filed on even date herewith, each of which is incorporated herein by reference.

The following examples are illustrative and are not intended as limiting.

### EXAMPLES

### Example 1

### Generation and Functional Analysis of mAb13.2

To generate an antibody that recognizes IL-13, polyclonal antisera were prepared by immunization of female BALB/c mice with recombinant human IL-13 (R&D Systems, Minneapolis, Minn.). Sera were screened for binding to human IL-13 by ELISA. Splenocytes from a mouse demonstrating high serum antibody titers were fused with the P3X63_AG8.653 myeloma cell line (ATCC, Manassas, Va.), and plated in selective media. Fusions were isolated with three rounds of subcloning by limiting dilution and screened for the production of antibodies that had a binding affinity to human IL-13. Three monoclonal antibodies were capable of binding IL-13 and neutralizing and/or inhibiting its bioactivity. The monoclonal antibody mAb13.2 (IgG1κ) was the subject of further analysis.

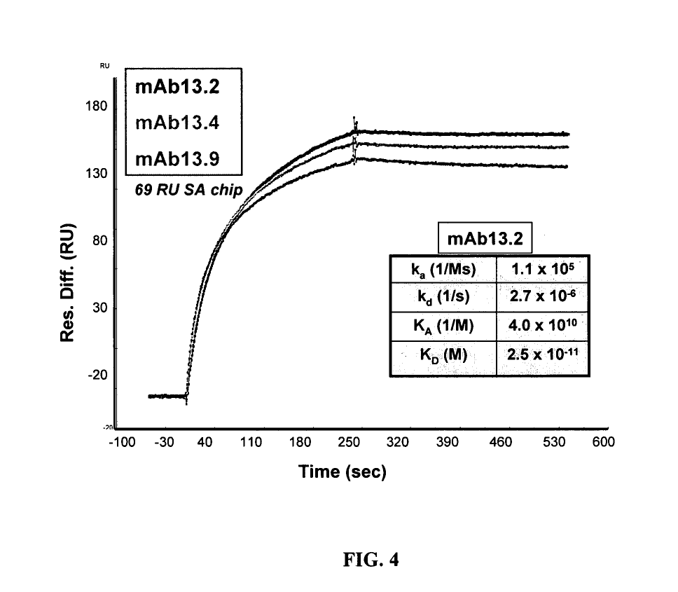

Several assays were performed to confirm that the murine monoclonal antibody mAb13.2 binds with high affinity and specificity to human IL-13. First, Biacore analysis confirmed that mAb13.2 had a rapid on-rate, slow off-rate, and high affinity for binding to human IL-13 (FIG. 4).

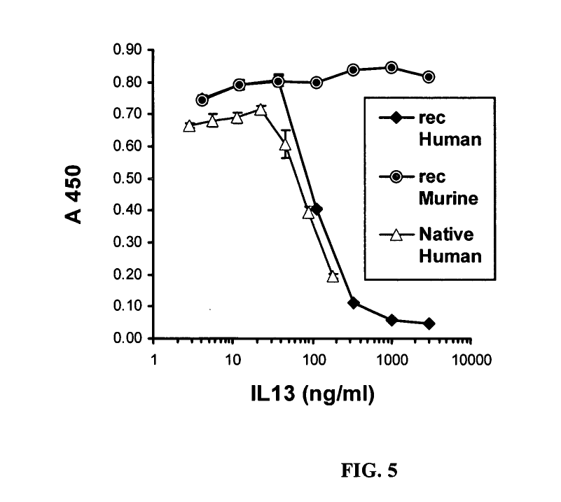

ELISA assays showed that mAb13.2 bound to all forms of human IL-13 tested, including native IL-13 derived from cord blood T cells (FIG. 5). To perform the assays with recombinant human IL-13, ELISA plates were coated with anti-FLAG M2 antibody. The binding of recombinant FLAG-tagged human IL-13 was detected with biotinylated mAb13.2 and streptavidin-peroxidase. This binding could be competed with native human IL-13 isolated from mitogen activated, TH2-skewed, cord blood mononuclear cells and with recombinant human IL-13 (FIG. 5). Recombinant murine IL-13 could not compete for binding with mAb13.2. Unlabeled mAb13.2 and unlabeled mAb13.2 Fab were also able to compete for binding to the flag-tagged IL-13 with biotinylated mAb13.2 (FIG. 11A). The IL-13 specific nornneutralizing monoclonal antibody mAb13.8 could not compete with biotinylated mAb13.2 binding.

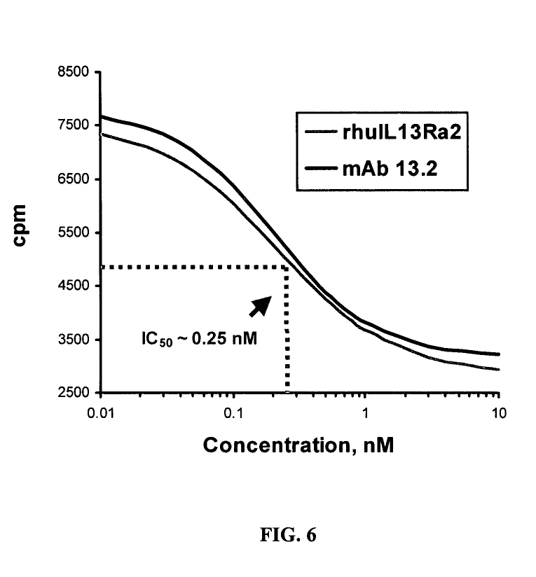

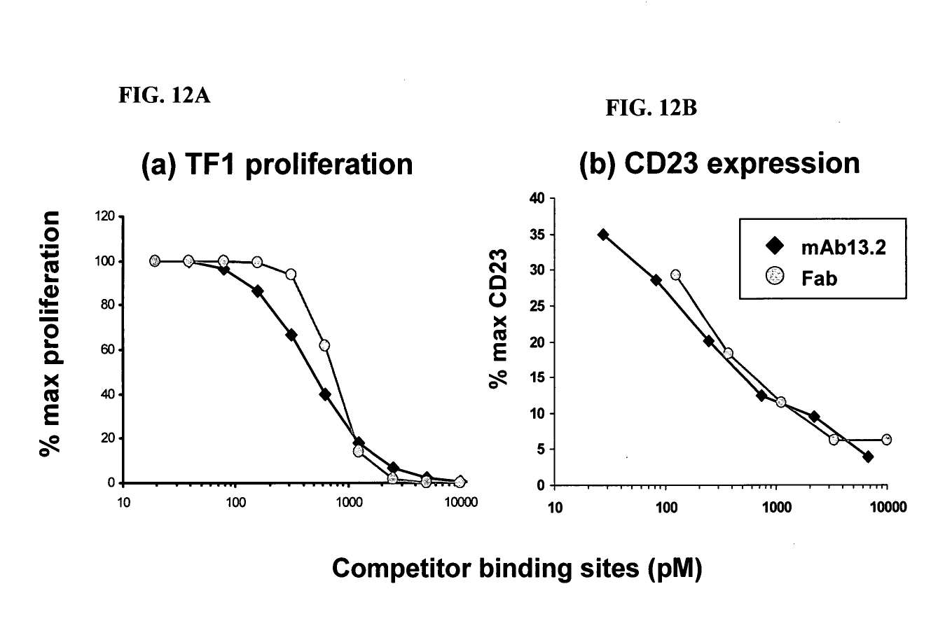

The ability of mAb13.2 to neutralize IL-13 bioactivity in vitro was confirmed using a TF1 bioassay, human peripheral blood monocytes, and human peripheral blood B cells. In the presence of suboptimal concentrations of IL-13, the proliferation of cells of the human erythroleukemic TF1 cell line can be made IL-13-dependent. The TF1 cell line was starved for cytokine, then exposed to a suboptimal concentration (3 ng/mL) of recombinant human IL-13 in the presence of varying concentrations of purified mouse mAb13.2 or the soluble inhibitor rhuIL-13Rα2. Cells were incubated for three days, and ³H-thymidine incorporation over the final four hours was determined by liquid scintillation counting. At suboptimal IL-13 concentrations (3 ng/mL), mAb13.2 caused a dose-dependant inhibition of TF1 proliferation (FIG. 6 and FIG. 12A). The IC₅₀ for this effect, 250 pM, is comparable to the IC₅₀ of rhuIL-13Rα2. The mAb13.2 Fab also inhibited CD23 expression human PBMCs.

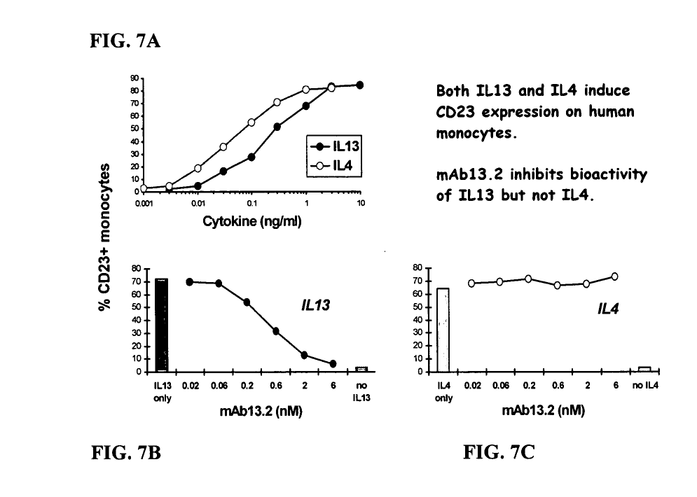

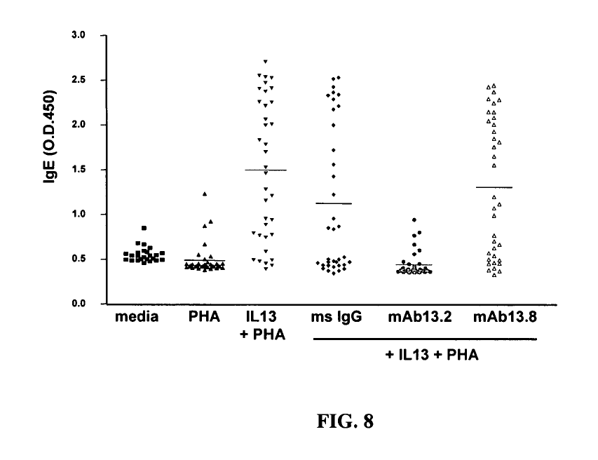

Human PBMCs respond to IL-13 or IL-4 by increasing cell-surface expression of low affinity IgE receptor (CD23) in a dose-dependent manner (see FIG. 7A). Monocytes (CD11b⁺) were therefore used to confirm the ability of mAb13.2 to neutralize IL-13 bioactivity. CD11b⁺ monocytes were treated for 12 hours with 1 ng/mL recombinant human IL-13 (FIG. 7B) or IL-4 (FIG. 7C) in the presence of the indicated concentration of purified mouse mAb13.2. Cells were then harvested and stained with CyChrome-labeled anti-CD11b antibodies and PE-labeled anti-CD23 antibodies. Labeling was detected by flow cytometry. The mAb13.2 inhibited IL-13-induced CD23 expression (FIG. 7B; see also FIG. 12B), but did not inhibit IL-4-induced CD23 expression (FIG. 7C).

The effects of mAb13.2 were also tested in a model of IL-13-induced IgE production by human peripheral blood B cells. In response to IL-13 and the T cell mitogen, phytohemaglutinin (PHA), human B cells undergo an Ig isotype switch recombination to IgE, resulting in higher IgE levels in culture. This effect can be seen as an increased frequency of IgE-producing B cells. To examine the effect of mAb13.2 on IL-13-dependent IgE production in B cells, PBMCs from a healthy donor were cultured in microtiter wells in the presence of autologous irradiated PBMC as feeders, and stimulated with PHA and IL-13. After 3 weeks, each well was assayed for IgE by ELISA. PHA+IL-13 increased the frequency of IgE-producing B cell clones. This effect was inhibited by mAb13.2, but not by mAb13.8 (binds IL-13 but does not neutralize), or by irrelevant mouse IgGmAb13.2 efficiently blocked this effect of IL-13 on cultured B cells (FIG. 8).

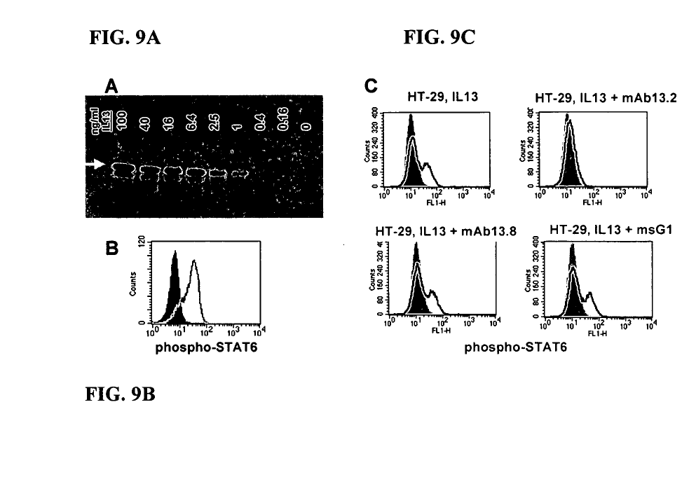

Finally, the ability of mAb13.2 to block an early cellular response to IL-13 was tested by examining effects on signal transducer and activator of transcription (STAT) 6 phosphorylation. Upon IL-13 interaction with its cell surface receptor, STAT6 dimerizes, becomes phosphorylated, and translocates from the cytoplasm to the nucleus, where it activates transcription of cytokine-responsive genes (Murata et al., J. Biol. Chem. 270:30829-36, 1995). Specific antibodies against phosphorylated STAT6 can detect this activation by Western blot or flow cytometry within 30 min of IL-13 exposure. To test the effect of mAb13.2 on IL-13 dependent STAT6 phosphorylation, cells of the HT-29 human epithelial cell line were treated with the indicated concentration of IL-13 for 30 minutes at 37° C. Phospho-STAT6 was detected in cell lysates by Western blot (FIG. 9A) or by flow cytometry (FIGS. 9B and 9C). In the experiment illustrated in FIG. 9B, cells were treated with a saturating concentration of IL-13 for 30 minutes at 37° C. and then fixed, permeabilized, and stained with an Alexa-Fluor 488-labeled mAb against phospho-STAT6. In the experiment illustrated in FIG. 9C, cells were treated with a suboptimal concentration of IL-13 in the presence or absence of an antibody, fixed and stained as described above. Flow cytometry results revealed that mAb13.2 blocked STAT6 phosphorylation, whereas mAb13.8 and the control mouse IgG1 had no effect.

### Example 2

### Murine Monoclonal Antibody mAb13.2 Neutralizes IL-13 Bioactivity In Vivo

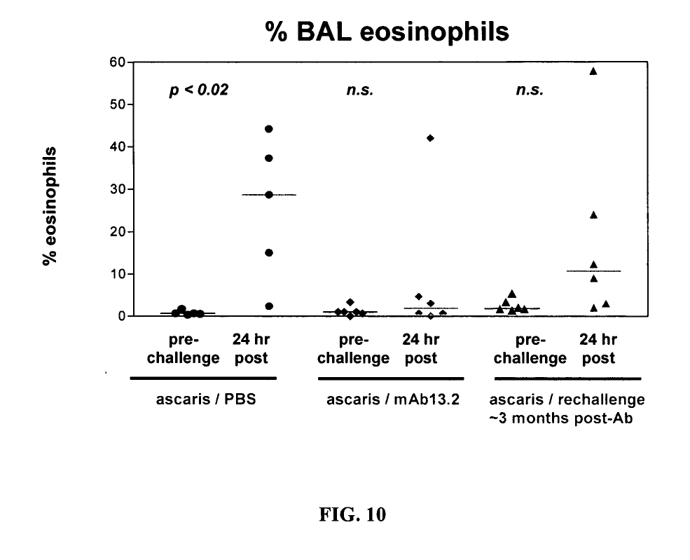

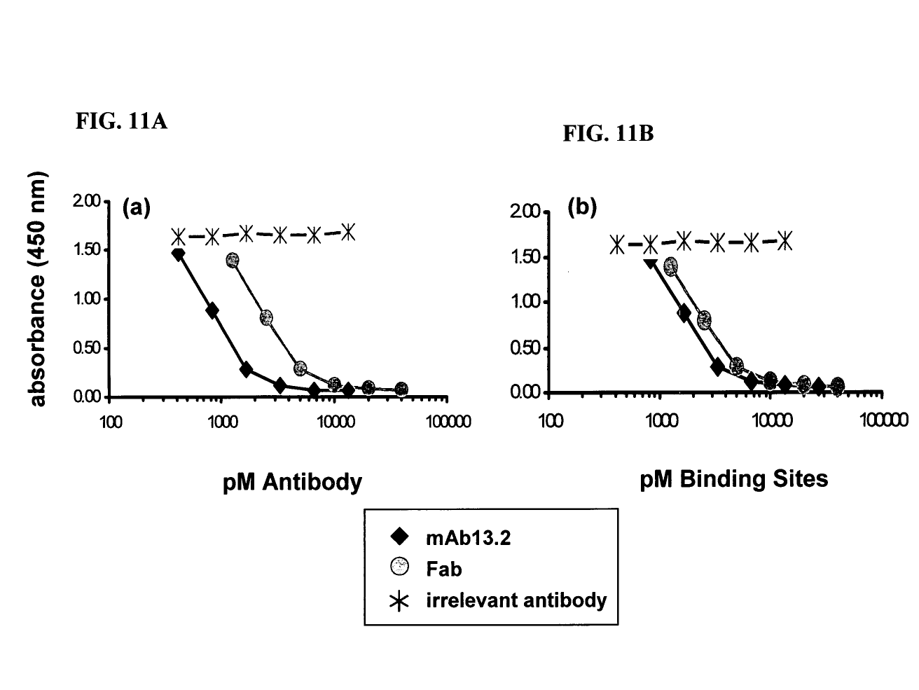

The ability of mouse mAb13.2 to neutralize IL-13 activity in vivo was tested using a model of antigen-induced airway inflammation in Cynomolgus monkeys naturally allergic to Ascaris suum. In this model, challenge of an allergic monkey with Ascaris suum antigen results in an influx of inflammatory cells, especially eosinophils, into the airways. To test the ability of mAb13.2 to prevent this influx of cells, the antibody was administered 24 hours prior to challenge with Ascaris suum antigen. On the day of challenge, a baseline lavage sample was taken from the left lung. The antigen was then instilled intratracheally into the right lung. Twenty-four hours later, the right lung was lavaged, and the bronchial alveolar lavage (BAL) fluid from animals treated intravenously with 8 mg/kg ascites purified mAb13.2 were compared to BAL fluid from untreated animals. Eosinophil counts increased in 4 of 5 untreated animals following challenge, as compared to 1 of 6 animals treated with mAb13.2 (FIG. 10). The percent BAL eosinophils was significantly increased for the untreated group (p<0.02), but not for the antibody-treated group. These results confirmed that mAb13.2 effectively prevents airway eosinophilia in allergic animals challenged with an allergen.

The average serum half-life of mouse mAb13.2 was less than one week in the monkeys. At the 3-month time point, when all traces of mAb13.2 would have been gone from the serum, mAb13.2-treated animals were rechallenged with Ascaris suum to confirm the Ascaris responsiveness of those individuals. Two of six monkeys in the treated group were found to be nonresponders.

### Example 3

### Murine Monoclonal Antibody mAb13.2 Binds to a Region of IL-13 that Normally Binds to IL-4Rα

IL-13 bioactivity is mediated through a receptor complex consisting of the IL-13Rα1 and IL-4Rα chains. The cytokine first undergoes a relatively low affinity interaction with IL-13Rα1 on the surface of cells. This complex then recruits IL-4Rα to form the high affinity receptor (Zurawski et al., EMBO J. 12:2663, 1993; Zurawski et al., J. Biol. Chem. 270:23869, 1995). Signaling through the IL-4Rα chain involves phosphorylation of STAT6, which can be monitored as one of the earliest cellular responses to IL-13 (Murata et al., J. Biol. Chem. 270:30829-36, 1995). Several approaches, such as epitope mapping, X-ray crystallography, and further Biacore analysis, were used to elucidate the interaction between murine mAb13.2 antibody and human IL-13, and further determine the basis for the IL-13 neutralizing effects of this antibody.

Epitope mapping and X-ray crystallography analysis indicated that mAb13.2 binds to the C-terminal region of IL-13 helix C, i.e., the IL-4R binding region (see below). To confirm this analysis, the interaction between mAb13.2 and IL-13 was analyzed with a Biacore chip. This analysis was done in several formats. First, IL-4R was bound to the Biacore chip, and a complex of IL-13 prebound to IL-13Rα1 was flowed over the chip. In the absence of mAb13.2, formation of a tri-molecular complex could be demonstrated. However, addition of mAb 13.2 to the mixture of IL-13 prebound to IL-13Rα1 prevented binding to IL-4R on the chip. Second, mAb13.2 was immobilized on the chip and bound IL-13 was added in solution phase. Although IL-13Rα1 was found to interact with the bound IL-13, no interaction of IL-4R with bound IL-13 was detected. Third, it was demonstrated that mAb13.2 could bind to IL-13 that was bound to IL-13Rα1-Fc or IL-13Rα1 monomer immobilized on the chip. These observations indicate that mAb13.2 does not inhibit IL-13 interaction with IL-13Rα1 but disrupts the interaction of IL-13Rα1 with IL-4Rα. This disruption is thought to prevent formation of the IL-13 signaling complex. These observations provided a model for the neutralization activity of this antibody.

The in vitro demonstration of a complex of mAb13.2 with IL-13 and IL-13Rα1 suggests that mAb13.2 could potentially be bound to receptor-associated IL-13 at the cell surface. In order to determine whether cell-bound mAb13.2 could be detected under conditions of saturating receptor-bound IL-13, the HT-29 human epithelial cell line was loaded with IL-13 at 4° C. and tested for antibody binding. No cell-bound mAb could be detected by flow cytometry. This observation, together with the demonstration that mAb 13.2 is a potent neutralizer of IL-13 bioactivity, indicated that normal functioning of the IL-13 receptor is disrupted by mAb13.2.

### Example 4

### Crystal Structure of Anti-IL-13 Antibody mAb13.2 Fab Fragment

Monoclonal antibody mAb13.2 from mouse ascites was purified using a Protein A affinity column. The mouse ascites was diluted 2× with Protein A binding buffer (50 mM Tris-HCl, 500 mM NaCl, pH 8.0) and filtered through a 0.2 mm filter unit. The filtered solution was applied to a Poros Protein A column (Applied Biosystems, Framingham, Mass.) equilibrated with the binding buffer at 4° C. The column was washed with the binding buffer, and the IgG was eluted using 100 mM Glycine (pH 3.0). The eluted IgG was neutralized immediately with 1M Tris-HCl at pH 8.0.

The Fab fragment was prepared by digesting the IL-13 monoclonal IgG with activated papain (Sigma, St. Louis, Mo.). Papain was activated by diluting the stock enzyme solution with the digestion buffer (50 mM Tris-HCl, 50 mM NaCl, 20 mM EDTA and 20 mM Cysteine, pH 7.5) on ice to give a final papain concentration of 1 mg/mL. Cleavage of IgG was performed by incubation with activated papain at a ratio of 100:1 w/w in papain digestion buffer for 7-8 hours at 37° C. The reaction was stopped by dialysis in 50 mM Tris-HCl (pH 7.5) overnight at 4° C. The dialyzed solution was loaded onto a tandem Poros HS/Protein A column equilibrated with 50 mM Tris-HCl (pH 7.5) at 4° C. to remove the papain and the Fc fragment. The flow-through of the tandem columns containing the Fab fragment was then loaded onto a hydroxylapatite column (Bio-Rad, Hercules, Calif.) equilibrated with 1 mM Sodium Phosphate and 20 mM Tris-HC1, pH 7.5, and eluted with a 1 mM to 125 mM Sodium Phosphate gradient at 25° C. The eluted Fab fragment solution was dialyzed overnight in 50 mM Tris-HCl (pH 8.0) at 4° C. After dialysis, the solution was loaded onto a Poros HQ column equilibrated with 50 mM Tris-HCl (pH 8.0). The flow-through was collected and ammonium sulfate was adjusted to a final concentration of 1.5 M before loading onto a Polypropyl Aspartamide column (Nest Group, Southborough, Mass.). The Fab fragment was eluted from the column with a 1.5 to 0 M ammonium sulfate gradient at 25° C. The protein was dialyzed in 50 mM Tris-HCl (pH 8.0) at 4° C.

The isolated mAb13.2 antibody and mAb13.2 Fab fragment were tested for their ability to inhibit IL-13 bioactivity. In one assay, purified mAb13.2 and mAb13.2 Fab fragment were tested for their ability to compete for binding with biotinylated mAb13.2 in an ELISA assay. ELISA plates were coated with anti-FLAG M2 antibody. The binding of FLAG-human IL-13 was detected with biotinylated mAb13.2 and streptavidin-peroxidase. Both the intact antibody and the Fab fragment were able to compete for binding, while the IL-13-specific nonneutralizing antibody mAb13.8 could not compete for binding (FIGS. 11A and 11B).

In other assays, purified mAb13.2 and mAb13.2 Fab fragment were tested for their ability to inhibit IL-13-dependent TF1 cell proliferation and IL-13-dependent CD23 expression on PBMCs. TF1 cells were incubated with 3 ng/mL recombinant human IL-13 as described in Example 1. The cells were treated with increasing concentrations of purified mAb13.2 or mAb13.2 Fab, and cell proliferation was monitored as described. Both the intact antibody and the Fab fragment inhibited IL-13-dependent TF1 cell proliferation (FIG. 12A). To test the effect of the isolated proteins on CD23 expression, PBMCs were incubated with 1 ng/mL recombinant human IL-13 as described in Example 1. The monocytes were treated with increasing concentrations of mAb13.2 or mAb13.2 Fab, and CD23 expression was monitored by flow cytometry as described above. The purified intact antibody and the purified Fab fragment were each capable of inhibiting IL-13-dependent CD23 expression (FIG. 12B).

For crystallization, purified mAb13.2 Fab was prepared at a concentration of 12.6 mg/mL in a solution of 50 mM Tris (pH 8.0) and 50 mM NaCl. One microliter of protein solution was mixed with 1 μl of crystallization solution (20% PEG 3350, 200 mM K₂SO₄) (Hampton Research, Aliso Viejo, Calif.), and the crystals formed at about 18° C. by the hanging drop method of vapor diffusion.

Data from crystals for the mAb13.2 Fab fragment were collected on beamline 5.0.2 at the Advanced Light Source (ALS) (Berkley, Calif.) using an ADSC Quantum-4 CCD detector. A single crystal, vitrified at −180° C., was used for each data set. The data were processed using DENZO and Scalepack (Otwinowski and Minor, Methods Enzymol. 276: 307-326, 1997) and the statistics from data collection and data refinement are shown in Tables 1 and 2 below, respectively.

TABLE 1
Statistics for Data Collection and Phase Determination

| Data Collection | mAb13.2 Fab | mAb13.2/IL-13 Fab |
|---|---|---|
| Crystal system | Orthorhombic | Cubic |
| Space group | P2₁2₁2₁ | P2₁3 |
| Unit cell dimensions | a = 54.442, | a = b = c = 125.261, |
| | b = 97.961, | α = β = γ = 90.0° |
| | c = 108.469, | |
| | α = β = γ = 90.0° | |
| Data collection temperature | −180° C. | −180° C. |
| Number of crystals | 1 | 1 |
| Radiation Source | ALS, Berkeley, CA | ALS, Berkeley, CA |
| Wavelength (Å) | λ = 1.0 Å | λ = 1.0 Å |
| Resolution range (Å) | 50-2.8 Å | 50-1.8 Å |
| Maximum resolution (Å) | 2.8 Å | 1.8 Å |
| R_{merge}^{a}(%) | 8.2% (38.4%) | 6.7% (48.6%) |
| % complete | 100% (100%) | 99.9% (99.0%) |
| total reflections | 98,254 | 561,539 |
| unique reflections | 14,903 | 57,656 |
| I/σ(I) | 23.3 (4.8) | 26.6 (2.8) |

^{a}R_{merge} = Σ|Iₕ − <Iₕ>|/ΣIₕ, where <Iₕ> is the average intensity over symmetry equivalents.
Number in parentheses reflects statistics for the last shell.

TABLE 2
Structure Refinement Statistics

| Data Collection | mAb13.2Fab | mAb13.2 Fab/IL-13 |
|---|---|---|
| Model for molecular | 2E8 Fab (12E8.pdb) | mAb13.2 Fab; soln. |
| replacement | | structure of IL-13^{b} |
| Maximum Resolution (Å) | 2.8 Å | 1.8 Å |
| R_{work}^{a}(%) | 25.9% | 20.3% |
| R_{free}(%) | 30.7% | 23.5% |

^{a}R_{work} = Σ||F_{obs}| − |F_{calc}||/Σ|F_{obs}|, R_{free} is equivalent to R_{work}, but calculated for a randomly chosen 5% of reflections that are omitted from the refinement process.
^{b}Moy et al., J. Mol. Biol. 310: 219-230, 2001.

The structure of mAb13.2 Fab was solved by molecular replacement using the program AMORE (Navaza, Acta Crystallogr. A50:157-163, 1994). The structure of the monoclonal 2E8 Fab antibody fragment (PDB code 12E8) was used as the probe. Prior to refinement, 5% of the data were randomly selected and designated as an R_{free} test set to monitor the progress of the refinement. The structure of the mAb 13.2 Fab was then rebuilt within QUANTA (Accelrys, San Diego, Calif.) utilizing a series of omit maps. Following six cycles of refinement with CNS (Brunger et al., Acta Crystallogr. D54: 905-921, 1998) and rebuilding using QUANTA, the refinement converged with a model that contained the mAb13.2 Fab and 41 water molecules at an R_{cryst} of 25.9% and an R_{free} of 30.7%. The structure refinement statistics are shown in Table 2. The crystal structure coordinates are shown in Table 10.

### Example 5

### Crystal Structure of mAb13.2 Fab/IL-13 Complex

Recombinant IL-13 (Swiss-Prot Accession Number P35225) and mAb13.2 Fab were purified for crystallization. Recombinant IL-13 was purified as follows. E. coli K12 strain G1934 was used for expression of Human IL-13. GI934 is an i/vg derivative of G1724 (LaVallie et al., Bio/Technology 11:187-193, 1993) that contains specific deletions in the two E. coli proteases ompT and ompP. Specifically, this strain contains the bacteriophage 1 repressor (cI) gene stably integrated into the chromosomal ampC locus. The cI gene is transcriptionally regulated by a synthetic Salmonella typhimurium trp promoter. E. coli expression vector pAL-981, a derivative of pAL-781 (Collins-Racie, et al., Bio/Technology 13:982-987, 1995), was used as the basis for construction of a Human IL-13 expression vector.

A cDNA of the human IL-13 gene was generated from synthetic oligonucleotide duplexes designed to possess silent changes from human IL-13 cDNA (Accession number NM_{—}002188) that was optimized for E. coli codon usage and increased AT content at the 5′ end of the gene. Three sets of complementary duplexes of synthetic oligonucleotides corresponding to amino acids Gly21 to Asn132 of the human IL-13 amino acid (SEQ ID NO:3) (FIG. 2A) were used to construct the mature region of human IL-13, which is the amino acid sequence of processed IL-13 (SEQ ID NO:4). The E. coli optimized complementary oligonucleotides of duplex 1 were

| | |
|---|---|
| 5′-TATGGGTCCAGTTCCACCATCTACTGCTCTGCG | (SEQ ID NO:6) |
| TGAACTGATTGAAGAACTGGTTAACATCACCCAGAA | |
| CCAGAAAGCTCCGCTGTGTAACGGTTCCATGGTTTG | |
| GTCCATCAACCTG-3′ | |
| with complement | |
| 5′-CAGCGGTCAGGTTGATGGACCAAACCATGGAAC | (SEQ ID NO:7) |
| CGTTACACAGCGGAGCTTTCTGGTTCTGGGTGATGT | |
| TAACCAGTTCTTCAATCAGTTCACGCAGAGCAGTAG | |
| ATGGTGGAACTGGACCCA-3; | |
| duplex 2 were | |
| 5′-ACCGCTGGTATGTACTGTGCAGCTCTGGAATCC | (SEQ ID NO:8) |
| CTGATCAACGTTTCTGGTTGCTCTGCTATCGAAAAA | |
| ACCCAGCGTATGCTGTCTGGTTTCTGCCCGCACAAA | |
| GTTTCCGCTGGTCAG-3′ | |
| with complement | |
| 5′-GAGGAGAACTGACCAGCGGAAACTTTGTGCGGG | (SEQ ID NO:9) |
| CAGAAACCAGACAGCATACGCTGGGTTTTTTCGATA | |
| GCAGAGCAACCAGAAACGTTGATCAGGGATTCCAGA | |
| GCTGCACAGTACATAC-3′; | |
| and duplex 3 were | |
| 5′-TTCTCCTCTCTGCACGTTCGTGACACCAAAATC | (SEQ ID NO:10) |
| GAAGTTGCTCAGTTCGTAAAAGACCTGCTGCTGCAC | |
| CTGAAAAAACTGTTCCGTGAAGGTCGTTTCAACTAA | |
| TAAT-3′ | |
| with complement | |
| 5′-CTAGATTATTAGTTGAAACGACCTTCACGGAAC | (SEQ ID NO:11) |
| AGTTTTTTCAGGTGCAGCAGCAGGTCTTTTACGAAC | |
| TGAGCAACTTCGATTTTGGTGTCACGAACGTGCAG | |
| A-3′. | |

The complement (bottom) strand of the first and second duplexes and the top strand of the second and third duplexes were phosphorylated independently. The complementary strands were combined, and each duplex mix was heated to 90° C. and then slowly cooled to allow annealing of the duplexes. The first and last duplexes respectively encoded the restriction endonucleases NdeI and XbaI to allow for cloning into an NdeI, XbaI digested and gel purified expression vector pAL-981. All restriction digests, enzymatic phosphorylation of oligonucleotides, DNA fragment isolations and ligations were carried out as described in Sambrook et al., 1989. “Molecular Cloning, a Laboratory Manual, second edition,” Cold Spring Harbor Laboratory Press, Cold Spring Harbor, N.Y. Ligation mixtures were transformed into electrocompetent G1934 as described (LaVallie et al., Methods Mol. Biol. 205:119-140, 2003). Ligation of the three sets of oligonucleotide duplexes into pAL-981 created plasmid pALHIL13-981. All synthetic oligonucleotides were sequence confirmed after cloning into the expression vector.

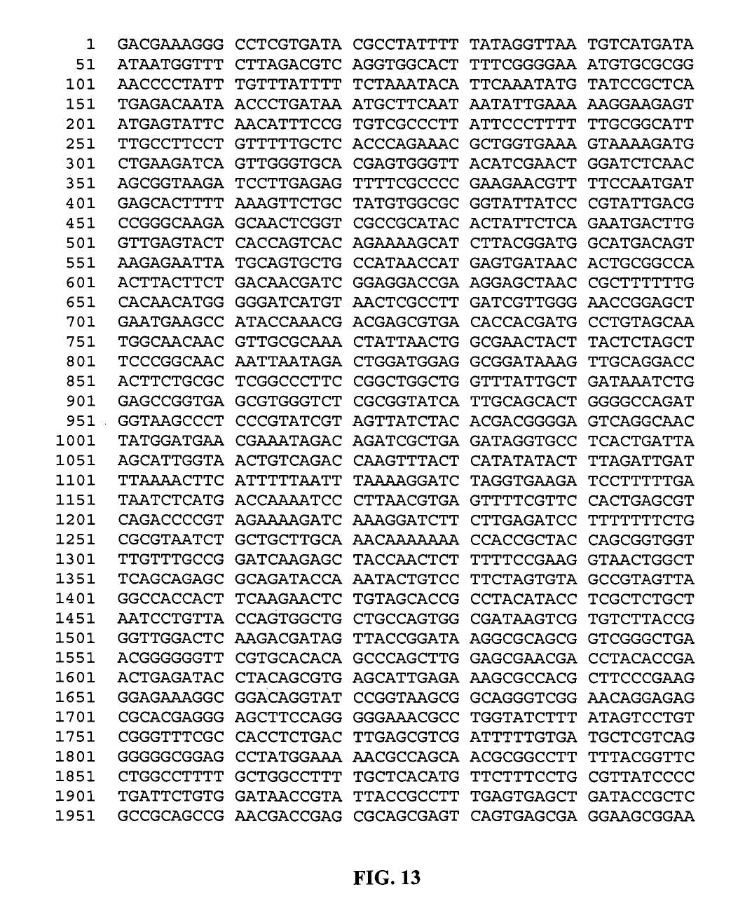

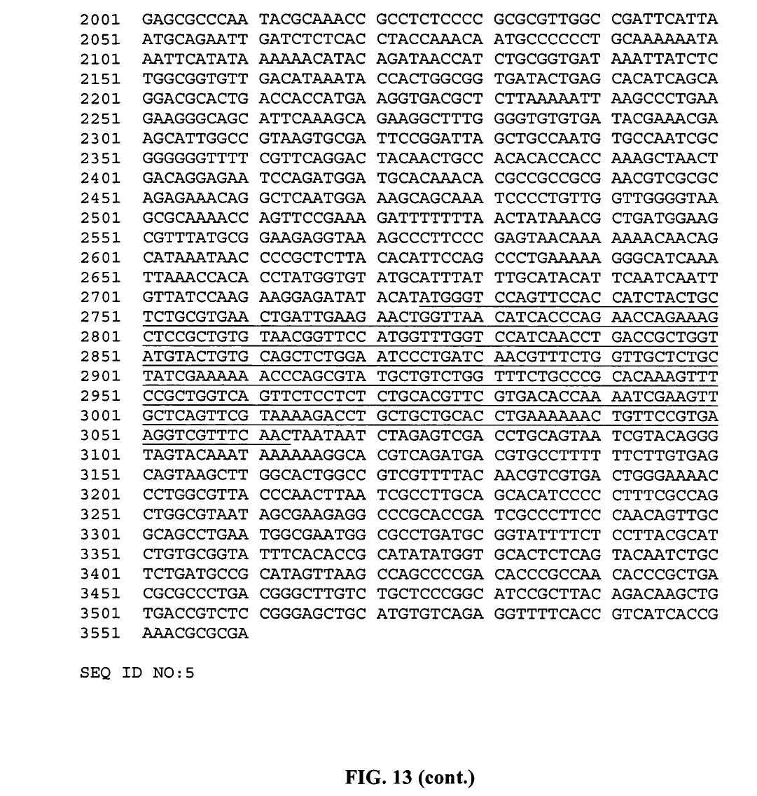

The resulting plasmid pALHIL13-981 was transformed into G1934. Optimal growth temperature of the culture for production of human IL-13 from plasmid pALHIL13-981 was determined empirically. Fermentor medium consisted of 1% casamino-acids, 1.75% w/v glucose, 50 mM KH₂PO₄, 15 mM (NH₄)₂SO₄, 30 mM Na₃.citrate.2H₂O, 20 mM MgSO₄, 100 μg/ml ampicillin, DM trace metals (300 μM FeCl₃, 29 μM ZnCl₃, 36 μM CoCl₂, 25 μM Na₂MoO₄, 20 μM CaCl₂, 22 μM CuCl₂, 24 μM H₃BO₃), and was adjusted to pH 7 with NH₄OH. A 10L fermentor was inoculated to A₅₅₀ 0.00005 with a fresh culture of G1934 containing pALHIL13-981 grown in Fermentor medium at 30° C. The fermentor culture was grown at 30° C. to A₅₅₀ of 1.2, then the temperature was adjusted to 37° C., and the culture was allowed to grow to A₅₅₀ of 7.5. Induction of protein synthesis from the pL promoter was initiated and the culture with the addition of tryptophan to 500 μg/ml. The culture was grown at 37° C. for 4.25 hours before harvesting the cells by centrifugation. The sequence of the expression vector is shown in FIG. 13 (SEQ ID NO:5).

The protein was essentially completely insoluble. Cells were broken with a microfluidizer and insoluble IL-13 was collected and dissolved at about 2 mg/mL in 50 mM Ches (pH 9), 6 M Guanidine-HCl, 1 mM EDTA, 20 mM DTT. The solution was diluted 20-fold into 50 mM Ches (pH 9), 3 M guanidine-HCl, 100 mM NaCl, 1 mM oxidized glutathione, and dialyzed twice against ten volumes of 20 mM Mes (pH 6). Following clarification by centrifugation, IL-13 was adsorbed to SP-Sepharose and eluted with a gradient of NaCl in Mes buffer. Final purification was by size-exclusion chromatography in 40 mM sodium phosphate, 40 mM NaCl on Superdex 75.

The mAb13.2 Fab was purified as described in Example 4.

The Fab:IL-13 complex was prepared by combining the two in a molar ratio of about 1:1. IL-13 (50 μM in 40 mM MES and 40 mM NaCl, pH 6.0) and mAb13.2 Fab (50 μM in 50 mM Tris.HCl, pH 8.0) were mixed together to give a final complex concentration of 50 μM. The complex was further purified by a Superdex 75 size exclusion column (Amersham Biosciences, Piscataway, N.J.) equilibrated with 50 mM Tris-HCl and 300 mM NaCl, pH 8.0, at 25° C. The purified complex was dialyzed in 50 mM Tris-HCl and 50 mM NaCl, pH 8.0, before setting up the crystallization.

For crystallization, purified mAb 13.2 Fab/II-13 complex was prepared at a concentration of 11.3 mg/mL in a solution of 50 mM Tris (pH 8.0) and 50 mM NaCl. One microliter of protein solution was mixed with 1 μl of crystallization solution (20% PEG 3350, 50 mM ZnOAc) (Hampton Research, Aliso Viejo, Calif.). The crystals formed at 18° C. by vapor diffusion by the hanging drop method.

Data from the crystal of the mAb 13.2 Fab/IL-13 complex were collected on beamline 5.0.2 at the ALS (Berkley, Calif.) using an ADSC Quantum-4 CCD detector. A single crystal, vitrified at −180° C., was used for the data set. The data were processed using DENZO and Scalepack (Otwinowski and Minor, Methods Enzymol. 276: 307-326, 1997). The statistics from data refinement are shown in Table 2. The crystal sructure coordinates are shown in Table 11.

Crystals of the binary mAb13.2Fab/IL-13 complex diffracted to 1.8 Å using synchrotron radiation. The structure of the complex was solved by molecular replacement using the program AMORE, and using the crystal structure of the mAb 13.2 Fab (described in Example 4) as the probe. Prior to refinement, 5% of the data were randomly selected and designated as an R_{free} test to monitor the progress of the refinement. This structure of the mAb13.2 Fab was then rebuilt within QUANTA using a series of omit maps. During this process, extra density was observed near the hypervariable regions, and these regions sharpened after each cycle of rebuilding. After the Fab fragment had been rebuilt, the NMR structure of IL-13 (Moy et al., J. Mol. Biol. 310:219-230, 2001) was rotated into the density adjacent to the hypervariable regions. Following three cycles of refinement with CNS (Accelrys, San Diego, Calif.) and rebuilding within QUANTA, the refinement converged with a model that contained one molecule of the mAb13.2 Fab, one molecule of IL-13, one acetate molecule, three zinc ions, and 465 water molecules at an R_{cyst} of 20.3% and R_{free} of 23.5%. The refinement statistics are shown in Table 2.

In the mAb13.2/IL-13 crystalline complex, residues 1-211 of the Fab light chain were visible, while residues 212, 213, and 214 were not observed in the density. For the heavy chain, residues 1-127 and 133-210 were modeled into the density, and no density was observed for residues 128 to 132. For IL-13, residues 7-21, 26-78, and 81-109 were visible and residues 1-6, 22-25, 79, and 80 were disordered. Several residues modeled as smaller residues due to inadequate electron density X-ray experiments (see Table 5).

There were three zinc molecules from the crystallization buffer that were found bound in this structure. None of them were involved in interactions between the IL-13 and Fab molecules. Two of the zinc molecules were involved in contacts between molecules in the asymmetric unit and symmetry related copies of the proteins, and thus they were important for crystallization of this complex. Zinc1 was coordinated to Fab light chain residues Glu27 and Glu97, and residues Glu189 and His193 of a symmetry related copy of the light chain (amino acids numbered according to SEQ ID NO:1 (FIG. 1A)). Zinc2 was coordinated to IL-13 residues His84 and Asp87, and residues Asp98 and His 102 of a symmetry related copy of IL-13. Zinc3 was coordinated to IL-13 residues Glu12 and Glu15 with water molecules as other ligands (amino acids numbered according to SEQ ID NO:4 (FIG. 2B)).

The residues of IL-13 interacting with the mAb 13.2 Fab fragment were located at the C-terminal end of helix C (residues 68-74). FIG. 3 illustrates the interaction of the C alpha helix of IL-13 with the CDR loops of the antibody. Hydrogen bond interactions were observed to exist between the Fab and IL-13 residues Glu49, Asn53, Gly69, Pro72, His73, Lys74, and Arg86. The N-terminal tip of helix A was within van der Waals distances of the Fab fragment. These interactions are summarized in Tables 3 and 4.

TABLE 3
H-bond Interactions between IL-13 and Fab 13.2

| IL-13^{a} | | | Fab 13.2 | | | | |
|---|---|---|---|---|---|---|---|
| Residue | | atom | Residue | Chothia^{b} | SEQ ID^{c} | Atom | Distance |
| Glu | 49I | OE1 | Asn | 30AL | 31L | ND2 | 2.87 Å |
| Glu | 49I | OE2 | Tyr | 98H | 101H | OH | 2.69 |
| Glu | 49I | OE2 | Tyr | 99H | 102H | OH | 2.54 |
| Asn | 53I | OD1 | Lys | 30DL | 34L | NZ | 2.74 |
| Gly | 69I | O | Ser | 53H | 53H | N | 2.91 |
| Pro | 72I | O | Tyr | 98H | 101H | N | 3.10 |
| His | 73I | ND1 | Asp | 94L | 98L | OD1 | 2.87 |
| His | 73I | NE2 | Ser | 50H | 50H | OG | 2.75 |
| Lys | 74I | NZ | Asn | 30AL | 31L | OD1 | 2.95 |
| Lys | 74I | NZ | Asn | 92L | 96L | OD1 | 2.61 |
| Arg | 86I | NH1 | Tyr | 30BL | 32L | OH | 3.16 |
| Arg | 86I | NH2 | Tyr | 30BL | 32L | OH | 2.93 |

^{a}Amino acid residues are numbered according to the processed form of IL-13 (SEQ ID NO: 4). “I” indicates amino acid of IL-13.
^{b}Amino acid residues correspond to SEQ ID NO: 1 (for light chain residues, “L”) or SEQ ID NO: 2 (for heavy chain residues, “H”), and are numbered according to the Chothia numbering system (Al-Lazikani et al., Jour. Mol. Biol. 273: 927-948, 1997).
^{c}Amino acid residues are numbered according to the numbering of SEQ ID NO: 1 (for light chain residues, “L”) or SEQ ID NO: 2 (for heavy chain residues, “H”).

TABLE 4
van der Waals Type Interactions between IL-13 and Fab 13.2

| IL-13 | | Fab 13.2 | | | |
|---|---|---|---|---|---|
| Residue^{a} | | Residue | Chothia^{b} | SEQ ID^{c} | CDR |
| Ser | 7I | Ile | 30H | 30H | CDR-H1 |
| Thr | 8I | Ile | 30H | 30H | CDR-H1 |
| Ala | 9I | Ile | 30H | 30H | CDR-H1 |
| Ala | 9I | Ser | 53H | 53H | CDR-H2 |
| Glu | 12I | Ile | 30H | 30H | CDR-H1 |
| Glu | 12I | Ser | 31H | 31H | CDR-H1 |
| Leu | 48I | Tyr | 98H | 101H | CDR-H3 |
| Glu | 49I | Tyr | 98H | 101H | CDR-H3 |
| Glu | 49I | Asn | 30AL | 31L | CDR-L1 |
| Glu | 49I | Tyr | 99H | 102H | CDR-H3 |
| Ile | 52I | Tyr | 99H | 102H | CDR-H3 |
| Ile | 52I | Tyr | 99H | 102H | CDR-H3 |
| Ile | 52I | Tyr | 99H | 102H | CDR-H3 |
| Ile | 52I | Arg | 50L | 54L | CDR-L2 |
| Ile | 52I | Tyr | 99H | 102H | CDR-H3 |
| Ile | 52I | Lys | 30DL | 34L | CDR-L1 |
| Asn | 53I | Lys | 30DL | 34L | CDR-L1 |
| Asn | 53I | Lys | 30DL | 34L | CDR-L1 |
| Asn | 53I | Lys | 30DL | 34L | CDR-L1 |
| Arg | 65I | Phe | 100H | 103H | CDR-H3 |
| Arg | 65I | Asp | 96H | 99H | CDR-H3 |
| Met | 66I | Ser | 31H | 31H | CDR-H1 |
| Ser | 68I | Asp | 96H | 99H | CDR-H3 |
| Ser | 68I | Phe | 100H | 103H | CDR-H3 |
| Gly | 69I | Ser | 31H | 31H | CDR-H1 |
| Gly | 69I | Ala | 33H | 33H | CDR-H1 |
| Gly | 69I | Ser | 53H | 53H | CDR-H2 |
| Gly | 69I | Ser | 52H | 52H | CDR-H2 |
| Phe | 70I | Ser | 53H | 53H | CDR-H2 |
| Phe | 70I | Ser | 52H | 52H | CDR-H2 |
| Cys | 71I | Tyr | 98H | 101H | CDR-H3 |
| Pro | 72I | Ala | 33H | 33H | CDR-H1 |
| Pro | 72I | Leu | 95H | 98H | CDR-H3 |
| Pro | 72I | Ser | 52H | 52H | CDR-H2 |
| Pro | 72I | Tyr | 58H | 58H | CDR-H2 |
| Pro | 72I | Tyr | 98H | 101H | CDR-H3 |
| Pro | 72I | Gly | 97H | 100H | CDR-H3 |
| Pro | 72I | Trp | 96L | 100L | CDR-L3 |
| His | 73I | Asp | 94L | 98L | CDR-L3 |
| His | 73I | Trp | 96L | 100L | CDR-L3 |
| His | 73I | Trp | 47H | 47H | |
| His | 73I | Leu | 95H | 98H | CDR-H3 |
| His | 73I | Tyr | 58H | 58H | CDR-H2 |
| His | 73I | Ser | 50H | 50H | CDR-H2 |
| His | 73I | Tyr | 98H | 101H | CDR-H3 |
| Lys | 74I | Tyr | 98H | 101H | CDR-H3 |
| Lys | 74I | Asn | 30AL | 31L | CDR-L1 |
| Lys | 74I | Asn | 92L | 96L | CDR-L3 |
| Arg | 86I | Tyr | 30BL | 32L | CDR-L1 |

^{a}Amino acid residues are numbered according to the processed form of IL-13 (SEQ ID NO: 4). “I” indicates amino acid of IL-13.
^{b}Amino acid residues correspond to SEQ ID NO: 1 (for light chain residues, “L”) or SEQ ID NO: 2 (for heavy chain residues, “H”), and are numbered according to the Chothia numbering system (Al-Lazikani et al., Jour. Mol. Biol. 273: 927-948, 1997). See Tables 6 and 7.
^{c}Amino acid residues are numbered according to the numbering of SEQ ID NO: 1 (for light chain residues, “L”) or SEQ ID NO: 2 (for heavy chain residues, “H”).

TABLE 5
Residues mis-modeled due to inadequate electron density

| Coordinates | Protein^{a} | Chothia^{b} | SEQ ID^{c} | Sequence^{c} | Modeled As |
|---|---|---|---|---|---|
| Table 11 | LC | 45 | 49 | Lys | Ala |
| Table 11 | HC | 3 | 3 | Lys | Ala |
| Table 11 | HC | 105 | 110 | Gln | Ala |
| Table 11 | HC | 171 | 176 | Glu | Ala |
| Table 11 | HC | 177 | 182 | Leu | Ala |
| Table 11 | HC | 205 | 210 | Lys | Ala |
| Table 11 | I | | 89 | Lys | Ala |
| Table 11 | I | | 94 | Gln | Ala |
| Table 11 | I | | 97 | Lys | Ala |
| Table 11 | I | | 105 | Lys | Ala |
| Table 11 | I | | 108 | Arg | Ala |
| Table 11 | I | | 109 | Glu | Ala |

^{a}“HC” is heavy chain (SEQ ID NO: 2); “LC” is light chain (SEQ ID NO: 1); “I” is IL-13 processed (SEQ ID NO: 4).
^{b}Amino acid residues correspond to SEQ ID NO: 1 (for light chain residues, “LC”) or SEQ ID NO: 2 (for heavy chain residues, “HC”), and are numbered according to the Chothia numbering system (Al-Lazikani et al., Jour. Mol. Biol. 273: 927-948, 1997). See Tables 6 and 7.
^{c}Amino acid residues are numbered and identified according to the numbering of SEQ ID NO: 1 (for light chain residues, “LC”), SEQ ID NO: 2 (for heavy chain residues, “HC”), or SEQ ID NO: 4 (for residues of the IL-13 processed polypeptide, “I”).

FIG. 3 is a ribbon diagram illustrating the co-crystal structure of mAb13.2 Fab with human IL-13. The light chain of mAb13.2 Fab is shown in dark shading, and the heavy chain in light shading. The IL-13 structure is shown at right. The figure depicts the interaction of the C alpha helix of IL-13 with the CDR loops of the antibody. The major residues of mAb13.2 heavy chain that make hydrogen bond contacts with IL-13 are SER50 (CDR2), SER53 (CDR2), TYR101 (CDR3), and TYR102 (CDR3). The major residues of mAb 13.2 heavy chain that make van der Waals contacts with IL-13 are ILE30 (CDR1), SER31 (CDR1), ALA33 (CDR1), TRP47, SER50 (CDR2), SER52 (CDR2), SER53 (CDR2), TYR58 (CDR2), LEU98 (CDR3), ASP99 (CDR3), GLY100 (CDR3), TYR101 (CDR3), TYR102 (CDR3), and PHE103 (CDR3) (see Table 4; amino acids numbered according the numbering of SEQ ID NO:2 (FIG. 1B)).

According to the amino acid numbering of SEQ ID NO:1 (FIG. 1A), the major residues of mAb 13.2 light chain that make hydrogen bond contacts with IL-13 are ASN31 (CDR1), TYR32 (CDR1), LYS34 (CDR1), ASN96 (CDR3), and ASP98 (CDR3). The major residues of mAb13.2 light chain that make van der Waals contacts with IL-13 are ASN31 (CDR1), TYR32 (CDR1), LYS34 (CDR1), ARG54 (CDR2), ASN96 (CDR3), ASP98 (CDR3), and TRP 100 (CDR3) (see Table 4).

Various numbering schemes have evolved to describe the amino acid residues of the heavy and light chain polypeptides of an antibody. The Kabat and Chothia schemes number the amino acid residues linearly accept in the defined CDR region of the polypeptide, where insertions are noted. The Kabat system (Kabat et al., NIH Publ. No. 91-3242, 5^{th} ed., vols. 1-3, Dept. of Health and Human Services, 1991) defines the location of the heavy and light chain CDRs by sequence variability, while the Chothia system (Al-Lazikani et al., Jour Mol. Biol. 273:927-948, 1997) defines the location structurally by loop regions. Because of the different placement of the CDR insertions, the numbering of the amino acids in the heavy chain and light chain can vary between the two systems. The notation of amino acid insertions causes each of these numbering systems to deviate from the linear numbering. Tables 6 and 7 align the amino acid sequences of the light and heavy chains, respectively, of mAb13.2Fab according to these three different numbering schemes (Kabat, Chothia, and linear numbering).

TABLE 6
Amino acid sequence of the light chain of mAb13.2Fab
according to the linear (SEQ ID NO: 1), Chothia
and Kabat numbering systems.^{a}

| | Linear | Chothia | Kabat |
|---|---|---|---|
| | Sequence | Structure | Sequence |
| Residue | Number | Number | Number |
| D | 1 | 1 | 1 |
| I | 2 | 2 | 2 |
| V | 3 | 3 | 3 |
| L | 4 | 4 | 4 |
| T | 5 | 5 | 5 |
| Q | 6 | 6 | 6 |
| S | 7 | 7 | 7 |
| P | 8 | 8 | 8 |
| A | 9 | 9 | 9 |
| S | 10 | 10 | 10 |
| L | 11 | 11 | 11 |
| A | 12 | 12 | 12 |
| V | 13 | 13 | 13 |
| S | 14 | 14 | 14 |
| L | 15 | 15 | 15 |
| G | 16 | 16 | 16 |
| Q | 17 | 17 | 17 |
| R | 18 | 18 | 18 |
| A | 19 | 19 | 19 |
| T | 20 | 20 | 20 |
| I | 21 | 21 | 21 |
| S | 22 | 22 | 22 |
| C | 23 | 23 | 23 |
| K | 24 | 24 | 24 |
| A | 25 | 25 | 25 |
| S | 26 | 26 | 26 |
| E | 27 | 27 | 27 |
| S | 28 | 28 | 27A |
| V | 29 | 29 | 27B |
| D | 30 | 30 | 27C |
| N | 31 | 30A | 27D |
| Y | 32 | 30B | 28 |
| G | 33 | 30C | 29 |
| K | 34 | 30D | 30 |
| S | 35 | 31 | 31 |
| L | 36 | 32 | 32 |
| M | 37 | 33 | 33 |
| H | 38 | 34 | 34 |
| W | 39 | 35 | 35 |
| Y | 40 | 36 | 36 |
| Q | 41 | 37 | 37 |
| Q | 42 | 38 | 38 |
| K | 43 | 39 | 39 |
| P | 44 | 40 | 40 |
| G | 45 | 41 | 41 |
| Q | 46 | 42 | 42 |
| S | 47 | 43 | 43 |
| P | 48 | 44 | 44 |
| K | 49 | 45 | 45 |
| L | 50 | 46 | 46 |
| L | 51 | 47 | 47 |
| I | 52 | 48 | 48 |
| Y | 53 | 49 | 49 |
| R | 54 | 50 | 50 |
| A | 55 | 51 | 51 |
| S | 56 | 52 | 52 |
| N | 57 | 53 | 53 |
| L | 58 | 54 | 54 |
| E | 59 | 55 | 55 |
| S | 60 | 56 | 56 |
| G | 61 | 57 | 57 |
| I | 62 | 58 | 58 |
| P | 63 | 59 | 59 |
| A | 64 | 60 | 60 |
| R | 65 | 61 | 61 |
| F | 66 | 62 | 62 |
| S | 67 | 63 | 63 |
| G | 68 | 64 | 64 |
| S | 69 | 65 | 65 |
| G | 70 | 66 | 66 |
| S | 71 | 67 | 67 |
| R | 72 | 68 | 68 |
| T | 73 | 69 | 69 |
| D | 74 | 70 | 70 |
| F | 75 | 71 | 71 |
| T | 76 | 72 | 72 |
| L | 77 | 73 | 73 |
| T | 78 | 74 | 74 |
| I | 79 | 75 | 75 |
| N | 80 | 76 | 76 |
| P | 81 | 77 | 77 |
| V | 82 | 78 | 78 |
| E | 83 | 79 | 79 |
| A | 84 | 80 | 80 |
| D | 85 | 81 | 81 |
| D | 86 | 82 | 82 |
| V | 87 | 83 | 83 |
| A | 88 | 84 | 84 |
| T | 89 | 85 | 85 |
| Y | 90 | 86 | 86 |
| Y | 91 | 87 | 87 |
| C | 92 | 88 | 88 |
| Q | 93 | 89 | 89 |
| Q | 94 | 90 | 90 |
| S | 95 | 91 | 91 |
| N | 96 | 92 | 92 |
| E | 97 | 93 | 93 |
| D | 98 | 94 | 94 |
| P | 99 | 95 | 95 |
| W | 100 | 96 | 96 |
| T | 101 | 97 | 97 |
| F | 102 | 98 | 98 |
| G | 103 | 99 | 99 |
| G | 104 | 100 | 100 |
| G | 105 | 101 | 101 |
| T | 106 | 102 | 102 |
| K | 107 | 103 | 103 |
| L | 108 | 104 | 104 |
| E | 109 | 105 | 105 |
| I | 110 | 106 | 106 |
| K | 111 | 107 | 107 |
| R | 112 | 108 | 108 |
| A | 113 | 109 | 109 |
| D | 114 | 110 | 110 |
| A | 115 | 111 | 111 |
| A | 116 | 112 | 112 |
| P | 117 | 113 | 113 |
| T | 118 | 114 | 114 |
| V | 119 | 115 | 115 |
| S | 120 | 116 | 116 |
| I | 121 | 117 | 117 |
| F | 122 | 118 | 118 |
| P | 123 | 119 | 119 |
| P | 124 | 120 | 120 |
| S | 125 | 121 | 121 |
| S | 126 | 122 | 122 |
| E | 127 | 123 | 123 |
| Q | 128 | 124 | 124 |
| L | 129 | 125 | 125 |
| T | 130 | 126 | 126 |
| S | 131 | 127 | 127 |
| G | 132 | 128 | 128 |
| G | 133 | 129 | 129 |
| A | 134 | 130 | 130 |
| S | 135 | 131 | 131 |
| V | 136 | 132 | 132 |
| V | 137 | 133 | 133 |
| C | 138 | 134 | 134 |
| F | 139 | 135 | 135 |
| L | 140 | 136 | 136 |
| N | 141 | 137 | 137 |
| N | 142 | 138 | 138 |
| F | 143 | 139 | 139 |
| Y | 144 | 140 | 140 |
| P | 145 | 141 | 141 |
| K | 146 | 142 | 142 |
| D | 147 | 143 | 143 |
| I | 148 | 144 | 144 |
| N | 149 | 145 | 145 |
| V | 150 | 146 | 146 |
| K | 151 | 147 | 147 |
| W | 152 | 148 | 148 |
| K | 153 | 149 | 149 |
| I | 154 | 150 | 150 |
| D | 155 | 151 | 151 |
| G | 156 | 152 | 152 |
| S | 157 | 153 | 153 |
| E | 158 | 154 | 154 |
| R | 159 | 155 | 155 |
| Q | 160 | 156 | 156 |
| N | 161 | 157 | 157 |
| G | 162 | 158 | 158 |
| V | 163 | 159 | 159 |
| L | 164 | 160 | 160 |
| N | 165 | 161 | 161 |
| S | 166 | 162 | 162 |
| W | 167 | 163 | 163 |
| T | 168 | 164 | 164 |
| D | 169 | 165 | 165 |
| Q | 170 | 166 | 166 |
| D | 171 | 167 | 167 |
| S | 172 | 168 | 168 |
| K | 173 | 169 | 169 |
| D | 174 | 170 | 170 |
| S | 175 | 171 | 171 |
| T | 176 | 172 | 172 |
| Y | 177 | 173 | 173 |
| S | 178 | 174 | 174 |
| M | 179 | 175 | 175 |
| S | 180 | 176 | 176 |
| S | 181 | 177 | 177 |
| T | 182 | 178 | 178 |
| L | 183 | 179 | 179 |
| T | 184 | 180 | 180 |
| L | 185 | 181 | 181 |
| T | 186 | 182 | 182 |
| K | 187 | 183 | 183 |
| D | 188 | 184 | 184 |
| E | 189 | 185 | 185 |
| Y | 190 | 186 | 186 |
| E | 191 | 187 | 187 |
| R | 192 | 188 | 188 |
| H | 193 | 189 | 189 |
| N | 194 | 190 | 190 |
| S | 195 | 191 | 191 |
| Y | 196 | 192 | 192 |
| T | 197 | 193 | 193 |
| C | 198 | 194 | 194 |
| E | 199 | 195 | 195 |
| A | 200 | 196 | 196 |
| T | 201 | 197 | 197 |
| H | 202 | 198 | 198 |
| K | 203 | 199 | 199 |
| T | 204 | 200 | 200 |
| S | 205 | 201 | 201 |
| T | 206 | 202 | 202 |
| S | 207 | 203 | 203 |
| P | 208 | 204 | 204 |
| I | 209 | 205 | 205 |
| V | 210 | 206 | 206 |
| K | 211 | 207 | 207 |
| S | 212 | 208 | 208 |
| F | 213 | 209 | 209 |
| N | 214 | 210 | 210 |
| R | 215 | 211 | 211 |
| N | 216 | 212 | 212 |
| E | 217 | 213 | 213 |
| C | 218 | 214 | 214 |

^{a}Bold font indicates an insertion in the linear sequence according to the Chothia or Kabat numbering system. Bold and underlined residue indicates an insertion as determined by X-ray data.

TABLE 7
Amino acid sequence of the heavy chain of mAb13.2Fab
according to the linear (SEQ ID NO: 2), Chothia
and Kabat numbering systems.^{a}

| | Linear | Chothia | Kabat |
|---|---|---|---|
| | Sequence | Structure | Sequence |
| Residue | Number | Number | Number |
| E | 1 | 1 | 1 |
| V | 2 | 2 | 2 |
| K | 3 | 3 | 3 |
| L | 4 | 4 | 4 |
| V | 5 | 5 | 5 |
| E | 6 | 6 | 6 |
| S | 7 | 7 | 7 |
| G | 8 | 8 | 8 |
| G | 9 | 9 | 9 |
| G | 10 | 10 | 10 |
| L | 11 | 11 | 11 |
| V | 12 | 12 | 12 |
| K | 13 | 13 | 13 |
| P | 14 | 14 | 14 |
| G | 15 | 15 | 15 |
| G | 16 | 16 | 16 |
| S | 17 | 17 | 17 |
| L | 18 | 18 | 18 |
| K | 19 | 19 | 19 |
| L | 20 | 20 | 20 |
| S | 21 | 21 | 21 |
| C | 22 | 22 | 22 |
| A | 23 | 23 | 23 |
| A | 24 | 24 | 24 |
| S | 25 | 25 | 25 |
| G | 26 | 26 | 26 |
| F | 27 | 27 | 27 |
| T | 28 | 28 | 28 |
| F | 29 | 29 | 29 |
| I | 30 | 30 | 30 |
| S | 31 | 31 | 31 |
| Y | 32 | 32 | 32 |
| A | 33 | 33 | 33 |
| M | 34 | 34 | 34 |
| S | 35 | 35 | 35 |
| W | 36 | 36 | 36 |
| V | 37 | 37 | 37 |
| R | 38 | 38 | 38 |
| Q | 39 | 39 | 39 |
| T | 40 | 40 | 40 |
| P | 41 | 41 | 41 |
| E | 42 | 42 | 42 |
| K | 43 | 43 | 43 |
| R | 44 | 44 | 44 |
| L | 45 | 45 | 45 |
| E | 46 | 46 | 46 |
| W | 47 | 47 | 47 |
| V | 48 | 48 | 48 |
| A | 49 | 49 | 49 |
| S | 50 | 50 | 50 |
| I | 51 | 51 | 51 |
| S | 52 | 52 | 52 |
| S | 53 | 53 | 53 |
| G | 54 | 54 | 54 |
| G | 55 | 55 | 55 |
| N | 56 | 56 | 56 |
| T | 57 | 57 | 57 |
| Y | 58 | 58 | 58 |
| Y | 59 | 59 | 59 |
| P | 60 | 60 | 60 |
| D | 61 | 61 | 61 |
| S | 62 | 62 | 62 |
| V | 63 | 63 | 63 |
| K | 64 | 64 | 64 |
| G | 65 | 65 | 65 |
| R | 66 | 66 | 66 |
| F | 67 | 67 | 67 |
| T | 68 | 68 | 68 |
| I | 69 | 69 | 69 |
| S | 70 | 70 | 70 |
| R | 71 | 71 | 71 |
| D | 72 | 72 | 72 |
| N | 73 | 73 | 73 |
| A | 74 | 74 | 74 |
| R | 75 | 75 | 75 |
| N | 76 | 76 | 76 |
| I | 77 | 77 | 77 |
| L | 78 | 78 | 78 |
| Y | 79 | 79 | 79 |
| L | 80 | 80 | 80 |
| Q | 81 | 81 | 81 |
| M | 82 | 82 | 82 |
| S | 83 | 82A | 82A |
| S | 84 | 82B | 82B |
| L | 85 | 82C | 82C |
| R | 86 | 83 | 83 |
| S | 87 | 84 | 84 |
| E | 88 | 85 | 85 |
| D | 89 | 86 | 86 |
| T | 90 | 87 | 87 |
| A | 91 | 88 | 88 |
| M | 92 | 89 | 89 |
| Y | 93 | 90 | 90 |
| Y | 94 | 91 | 91 |
| C | 95 | 92 | 92 |
| A | 96 | 93 | 93 |
| R | 97 | 94 | 94 |
| L | 98 | 95 | 95 |
| D | 99 | 96 | 96 |
| G | 100 | 97 | 97 |
| Y | 101 | 98 | 98 |
| Y | 102 | 99 | 99 |
| F | 103 | 100 | 100 |
| G | 104 | 100A | 100A |
| F | 105 | 100B | 100B |
| A | 106 | 101 | 101 |
| Y | 107 | 102 | 102 |
| W | 108 | 103 | 103 |
| G | 109 | 104 | 104 |
| Q | 110 | 105 | 105 |
| G | 111 | 106 | 106 |
| T | 112 | 107 | 107 |
| L | 113 | 108 | 108 |
| V | 114 | 109 | 109 |
| A | 115 | 110 | 110 |
| V | 116 | 111 | 111 |
| S | 117 | 112 | 112 |
| A | 118 | 113 | 113 |
| A | 119 | 114 | 114 |
| K | 120 | 115 | 115 |
| T | 121 | 116 | 116 |
| T | 122 | 117 | 117 |
| P | 123 | 118 | 118 |
| P | 124 | 119 | 119 |
| S | 125 | 120 | 120 |
| V | 126 | 121 | 121 |
| Y | 127 | 122 | 122 |
| P | 128 | 123 | 123 |
| L | 129 | 124 | 124 |
| A | 130 | 125 | 125 |
| P | 131 | 126 | 126 |
| G | 132 | 127 | 127 |
| S | 133 | 128 | 128 |
| A | 134 | 129 | 129 |
| A | 135 | 130 | 130 |
| Q | 136 | 131 | 131 |
| T | 137 | 132 | 132 |
| N | 138 | 133 | 133 |
| S | 139 | 134 | 134 |
| M | 140 | 135 | 135 |
| V | 141 | 136 | 136 |
| T | 142 | 137 | 137 |
| L | 143 | 138 | 138 |
| G | 144 | 139 | 139 |
| C | 145 | 140 | 140 |
| L | 146 | 141 | 141 |
| V | 147 | 142 | 142 |
| K | 148 | 143 | 143 |
| G | 149 | 144 | 144 |
| Y | 150 | 145 | 145 |
| F | 151 | 146 | 146 |
| P | 152 | 147 | 147 |
| E | 153 | 148 | 148 |
| P | 154 | 149 | 149 |
| V | 155 | 150 | 150 |
| T | 156 | 151 | 151 |
| V | 157 | 152 | 152 |
| T | 158 | 153 | 153 |
| W | 159 | 154 | 154 |
| N | 160 | 155 | 155 |
| S | 161 | 156 | 156 |
| G | 162 | 157 | 157 |
| S | 163 | 158 | 158 |
| L | 164 | 159 | 159 |
| S | 165 | 160 | 160 |
| S | 166 | 161 | 161 |
| G | 167 | 162 | 162 |
| V | 168 | 163 | 163 |
| H | 169 | 164 | 164 |
| T | 170 | 165 | 165 |
| F | 171 | 166 | 166 |
| P | 172 | 167 | 167 |
| A | 173 | 168 | 168 |
| V | 174 | 169 | 169 |
| L | 175 | 170 | 170 |
| E | 176 | 171 | 171 |
| S | 177 | 172 | 172 |
| D | 178 | 173 | 173 |
| L | 179 | 174 | 174 |
| L | 180 | 175 | 175 |
| T | 181 | 176 | 176 |
| L | 182 | 177 | 177 |
| S | 183 | 178 | 178 |
| S | 184 | 179 | 179 |
| S | 185 | 180 | 180 |
| V | 186 | 181 | 181 |
| T | 187 | 182 | 182 |
| V | 188 | 183 | 183 |
| P | 189 | 184 | 184 |
| S | 190 | 185 | 185 |
| S | 191 | 186 | 186 |
| P | 192 | 187 | 187 |
| R | 193 | 188 | 188 |
| P | 194 | 189 | 189 |
| S | 195 | 190 | 190 |
| E | 196 | 191 | 191 |
| T | 197 | 192 | 192 |
| V | 198 | 193 | 193 |
| T | 199 | 194 | 194 |
| C | 200 | 195 | 195 |
| N | 201 | 196 | 196 |
| V | 202 | 197 | 197 |
| A | 203 | 198 | 198 |
| H | 204 | 199 | 199 |
| P | 205 | 200 | 200 |
| A | 206 | 201 | 201 |
| S | 207 | 202 | 202 |
| S | 208 | 203 | 203 |
| T | 209 | 204 | 204 |
| K | 210 | 205 | 205 |
| V | 211 | 206 | 206 |
| D | 212 | 207 | 207 |
| K | 213 | 208 | 208 |
| K | 214 | 209 | 209 |
| I | 215 | 210 | 210 |

^{a}Bold font indicates an insertion in the linear sequence according to the Chothia or Kabat numbering system. Bold and underlined residue indicates an insertion as determined by X-ray data.

### Example 6

### Crystal Structure of the Trimeric Complex of Interleukin-13, I1-13 receptor α1, and the Binding Domain of the Inhibitory antibody mAb13.2 Fab

The extracellular domain (residues 27-342; see FIG. 14) of IL-13Rα1 was expressed with a 6xHis tag fused at the C-terminus (Aman et al., J. Biol. Chem. 271:29265-29270, 1996). Expression was performed in the yeast Pichia pastoris. The recombinant protein was purified to homogeneity by affinity chromatography over NiNTA-agarose (Qiagen) followed by anion exchange chromatography over HiTrap Q Sepharose HP (Pharmacia, Amersham Pharmacia Biotech, UK) and gel filtration chromatography over Superdex-75 (Pharmacia).

The human IL-13 (amino acid residues 1 to 113) (SEQ ID NO:4) was expressed and purified as described in example 5.

A complex containing IL-13 and IL-13Rα1 was was formed by mixing the receptor with a slight excess of IL-13. Following confirmation of complex formation by analytical size-exclusion chromatography, the complex was treated with endoglycosidase Hf (endoHf) (25,000 units/mL) for 90 minutes at 37° C. The deglycosylated complexes were applied to a concanavalin A (conA)-Sepharose column to remove protein with uncleaved oligosaccharides, and the remaining complexes were applied to a NiNTA column to remove EndoHf. The purified complexes were purified to homogeneity by gel filtration chromatography over Superdex-200 (GE Healthcare, formerly Amersham Biosciences, Piscatway, N.J.). Formation of 1:1 complexes of IL-13 and IL-13Rα1 was confirmed by native polyacrylamide gel electrophoresis and size exclusion chromatography prior to crystal screening.

mAb13.2 Fab was purified as described in example 4.

Crystals of a complex of IL-13, IL-13Rα1, and mAb13.2 Fab were grown at 18° C. by vapor diffusion in hanging drops containing 10 mg/ml protein complex, 13% PEG-MME 2000 and 100 mM HEPES (pH 7.0). Crystals appeared in several weeks, but did not reach maximal size for several months. The crystals had the symmetry of space group 14 with unit cell dimensions a=164.9 Å, b=164.9 Å, and c=74.8 Å. Prior to data collection, crystals were briefly transferred to 10% ethylene glycol plus mother liquor and flash cooled in liquid nitrogen. Throughout data collection, the crystal was maintained at 100K. Data were collected at the 5.0.1 beam line at the Advanced Light Source, Berkeley, California. Intensities were integrated and scaled using DENZO (Otwinowski and Minor, Methods Enzymol. 276:307-326, 1997) and SCALA (“CCP4,” Acta Cryst D50:760-763, 1994).

The structure was solved by molecular replacement using the coordinates of the mAb 13.2 Fab/IL-13 complex (Table 11). Initial phases were improved by solvent flattening using Solomon as implemented in CCP4 (“CCP4,” Acta Cryst D50:760-763, 1994). Rigid body refinement within CCP4 was used to obtain an initial model. Experimental maps with continuous density were obtained, and an initial model was constructed using QUANTA (Accelrys, Inc., San Diego, Calif.) and refined against data from 30 to 2.2 Å with CNS (Brunger et al., Acta Cryst. D54:905-921, 1998). The final refined model, which includes polypeptide chains of IL-13Rα1 (residues 6-314), mAb13.2 Fab (light chain residues 1-213 and heavy chain residues 1-213) and IL-13 (residues 6-112), as well as 123 water molecules, has a working R-value of 24.4% and a free R-value of 27.2%. Statistics for data collection and refinement are shown in Tables 8 and 9. There were no backbone torsion angles outside of the allowed regions of the Ramachandran plot. Structural figures were generated using PYMOL (DeLano, “The PYMOL Molecular Graphics System” (2002) DeLano Scientific, San Carlos, Calif.) and Ribbons (Carson, J. Appl. Cryst. 24:958-961, 1991). The structural coordinates are provided in Table 12. The following residues of IL-13Rα1 had no density beyond the C-beta atom and the coordinates for each were truncated to reflect that ambiguity: 81E, 93R, 104T, 105N, 111S, 1121, 122E, 124D, 150R, 151T, 157N, 165R, 168E, 169K, 174E, 195S, 196S, 197F, 305D, 306T, 339K, 110P, 200Q, 203Q, 2041, 209N, 212K, 213I, 214K, 240N, 279E, 284N, and 293N.

TABLE 8
Statistics for Data Collection and Phase Determination

| Data Collection | IL-13/mAb13.2 Fab/IL-13Rα1 |
|---|---|
| Crystal system | Tetragonal |
| Space group | I4 |
| Unit cell dimensions | a = b = 164.9 Å, c = 74.8 Å, |
| | α = β = γ = 90.0° |
| Data collection temperature | 100 K |
| Number of crystals | 1 |
| Radiation Source | ALS, Berkeley, CA |
| Wavelength (Å) | λ = 1.0 Å |
| Resolution range(Å) | 30-2.2 Å |
| Maximum resolution (Å) | 2.2 Å |
| R_{merge}^{a}(%) | 6.7% (48.6%) |
| % complete | 99.9% (99.0%) |
| total reflections (free) | 42298 (2110) |
| unique reflections | 40188 |
| I/σ(I) | 26.6 (2.8) |

^{a}R_{merge} = Σ | Iₕ − <Iₕ> | /ΣIₕ, where <Iₕ> is the average intensity over symmetry equivalents.
Number in parentheses reflects statistics for the last resolution shell (2.8 Å −2.7 Å).

TABLE 9
Structure Refinement Statistics

| Data Collection | IL-13/mAb13.2 Fab/IL-13Rα1 |
|---|---|
| Model for molecular replacement | mAb13.2 Fab/IL-13 |
| Maximum Resolution (Å) | 2.2 Å |
| R_{work}^{a} (%) | 24.4% |
| R_{free} (%) | 27.2% |

^{a}R_{work} = Σ||F_{obs}| − |F_{calc}||/Σ|F_{obs}|, R_{free} is equivalent to R_{work}, but calculated for a randomly chosen 6.4% of reflections that are omitted from the refinement process.

There are two points of substantial interaction between IL-13 and IL-13Rα1. One interaction is between Ig domain 1 and a portion of the loop connecting helices C and D of the cytokine while the other interaction is between Ig domain 3 of the receptor and helices A and D of IL-13 (see FIG. 15). The interaction between Ig domain 1 of IL-13Rα1 and IL-13 results in the formation of an extended beta sheet spanning the two molecules. Residues Thr88, Lys89, Ile90 and Glu91 of IL-13 (SEQ ID NO:4) form a beta strand that interacts with residues Lys76, Lys77, Ile78 and Ala79 of the receptor (SEQ ID NO: 12) (See FIG. 16). Additionally, the side chain of Met33 of IL-13 extends into a hydrophobic pocket that is created by the side chains of these adjoining strands.

The predominant feature of the interaction with Ig domain 3 is the insertion of a hydrophobic residue (Phe107) of IL-13 into a hydrophobic pocket in Ig domain 3 of the receptor IL-13Rα1. The hydrophobic pocket of IL-13Rα1 is formed by the side chains of residues Leu319, Cys257, Arg256 and Cys320 (FIG. 17). The interaction with Phe107 of IL-13 results in an extensive set of Van der Waals interactions between amino acid residues Ile254, Ser255, Arg256, Lys318, Cys320, and Tyr321 of IL-13Rα1 (SEQ ID NO:12) and amino acid residues Arg11, Glu12, Leu13, Ile14, Glu15, Lys104, Lys105, Leu106, Phe107 and Arg108 of IL-13 (SEQ ID NO:4) (See FIG. 17).

LENGTHY TABLE REFERENCED HERE
US20070048785A1-20070301-T00001
Please refer to the end of the specification for access instructions.

LENGTHY TABLE REFERENCED HERE
US20070048785A1-20070301-T00002
Please refer to the end of the specification for access instructions.

LENGTHY TABLE REFERENCED HERE
US20070048785A1-20070301-T00003
Please refer to the end of the specification for access instructions.

Other embodiments are in the claims.

LENGTHY TABLE
The patent application contains a lengthy table section. A copy of the table is available in electronic form from the USPTO web site (http://seqdata.uspto.gov/?pageRequest=docDetail&DocID=US20070048785A1). An electronic copy of the table will also be available from the USPTO upon request and payment of the fee set forth in 37 CFR 1.19(b)(3).

1.-64. (canceled)

65. A method comprising:
using a three-dimensional model of an antibody to design an agent that interacts with an IL-13 polypeptide,
wherein the antibody comprises an anti-IL-13 antibody or a Fab fragment of an anti-IL-13 antibody.

66. The method of claim 65, wherein the three-dimensional model comprises a CDR of the antibody.

67. The method of claim 65, wherein the antibody is a Fab fragment of an anti-IL-13 antibody.

68. The method of claim 65, wherein the antibody comprises a light chain polypeptide including the amino acid sequence of SEQ ID NO:1, and a heavy chain polypeptide including the amino acid sequence of SEQ ID NO:2.

69. The method of claim 65, wherein the antibody is mAb13.2.

70. The method of claim 65, wherein the antibody is an mAb13.2 Fab fragment.

71. The method of claim 65, wherein the three-dimensional model comprises structural coordinates of atoms of the antibody.

72. The method of claim 71, wherein the structural coordinates are experimentally determined coordinates.

73. The method of claim 65, wherein the three-dimensional model comprises structural coordinates of an atom selected from the group consisting of atoms of amino acids Asn31, Tyr32, Lys34, Arg54, Asn96, Asp98, and Trp100 as defined by the amino acid sequence of SEQ ID NO:1, and Ile30, Ser31, Ala33, Trp47, Ser50, Ser52, Ser53, Tyr58, Leu98, Asp99, Gly100, Tyr101, Tyr102, and Phe103 as defined by the amino acid sequence of SEQ ID NO:2.

74. The method of claim 65, wherein the agent binds a region of the IL-13 polypeptide that binds an IL-4R polypeptide in vivo.

75. The method of claim 74, wherein the IL-4R polypeptide is an IL-4Rα polypeptide.

76. The method of claim 65, wherein the three-dimensional model comprises an IL-13 polypeptide bound to the antibody.

77. The method of claim 76, wherein the three-dimensional model further comprises an IL-13Rα1 polypeptide bound to the IL-13 polypeptide.

78. A method comprising:
using a three-dimensional model of an IL-13 polypeptide to design an agent that interacts with the IL-13 polypeptide.

79. The method of claim 78, wherein the three-dimensional model further comprises an antibody bound to the IL-13 polypeptide, the antibody comprising an anti-IL-13 antibody or a Fab fragment of an anti-IL-13 antibody.

80. The method of claim 79, wherein the three-dimensional model comprises structural coordinates of atoms of the antibody.

81.-82. (canceled)

83. The method of claim 78, wherein the three-dimensional model comprises structural coordinates of atoms of the IL-13 polypeptide.

84. (canceled)

85. The method of claim 83, wherein the structural coordinates are according to Table 11 +/− a root mean square deviation for alpha carbon atoms of not more than 1.5 Å.

86. The method of claim 78, wherein the three-dimensional model comprises structural coordinates of an atom selected from the group consisting of atoms of amino acids Glu49, Asn53, Ser68, Gly69, Phe70, Cys71, Pro72, His73, Lys74, and Arg86 of the IL-13 polypeptide as defined by the amino acid sequence of SEQ ID NO:4.

87. The method of claim 78, wherein the three-dimensional model further comprises an IL-13Rα1 polypeptide bound to the IL-13 polypeptide.

88. The method of claim 87, wherein the three-dimensional model comprises structural coordinates of atoms of the IL-13Rα1 polypeptide.

89. A method comprising:
using a three-dimensional model of an IL-13 polypeptide bound to an IL-13Rα1 polypeptide to design an agent that interacts with the IL-13 polypeptide.

90. The method of claim 89, wherein the three-dimensional model further comprises an antibody bound to the IL-13 polypeptide, the antibody comprising an anti-IL-13 antibody or a Fab fragment of an anti-IL-13 antibody.

91. (canceled)

92. The method of claim 89, wherein the three-dimensional model comprises structural coordinates of atoms of the IL-13 polypeptide and the IL-13Rα1 polypeptide.

93. (canceled)

94. The method of claim 92, wherein the structural coordinates are according to Table 12 +/− a root mean square deviation for alpha carbon atoms of not more than 1.5 Å.

95. A method, comprising:
selecting an agent by performing rational drug design with a three-dimensional structure of a crystalline complex that comprises an IL-13 polypeptide;
contacting the agent with an IL-13 polypeptide; and
detecting the ability of the agent to bind the IL-13 polypeptide.

96. The method of claim 95, wherein the crystalline complex of the three-dimensional structure further comprises an antibody bound to the IL-13 polypeptide, the antibody comprising an anti-IL-13 antibody or a Fab fragment of an anti-IL-13 antibody.

97. (canceled)

98. The method of claim 95, wherein the agent is selected via computer modeling.

99. The method of claim 95, wherein the three-dimensional structure comprises structural coordinates of Table 11, ±a root mean square deviation for alpha carbon atoms of not more than 1.5 Å.

100. The method of claim 95, wherein the crystalline complex of the three-dimensional structure further comprises an IL-13Rα1 polypeptide bound to the IL-13 polypeptide.

101. The method of claim 86, wherein the three-dimensional structure comprises structural coordinates of Table 12, ±a root mean square deviation for alpha carbon atoms of not more than 1.5 Å.

102.-166. (canceled)
